(19) United States
(12) Patent Application Publication
(10) Pub. No.: US 2025/0375484 A1
(43) Pub. Date: Dec. 11, 2025

(54) Vascular associated naturally pluripotent stem cell and method of isolation

(71) Applicant: SCICOTEC GMBH, GRUENWALD (DE)

(72) Inventor: Eckhard U. ALT, Houston, TX (US)

(21) Appl. No.: 19/067,665

(22) Filed: Feb. 28, 2025

Related U.S. Application Data
(62) Division of application No. 17/929,813, filed on Sep. 6, 2022, now abandoned.
(60) Provisional application No. 63/240,435, filed on Sep. 3, 2021.

(51) Int. Cl. A61K 35/545; A61P 19/04; C12N 5/0775

(52) U.S. Cl. CPC A61K 35/545; A61P 19/04; C12N 5/0667

(57) ABSTRACT

A composition comprising isolated vascular-associated naturally pluripotent stem cells (vaPS), is disclosed, as well as method of treating defects using such composition, wherein said vaPS are capable of differentiating into somatic cells of all three germ layers under the guidance of the respective microenvironment.

### PRIOR RELATED APPLICATIONS

This disclosure is a divisional application of U.S. application Ser. No. 17/929,813, filed on Sep. 6, 2022, which claims priority to U.S. Provisional App. No. 63/240,435, filed on Sep. 3, 2021, each of which is incorporated by reference in its entirety herein for all purposes.

### FEDERALLY SPONSORED RESEARCH STATEMENT

Not applicable.

### FIELD OF THE DISCLOSURE

The disclosure generally relates to vascular-associated pluripotent stem cells (vaPS), and more particularly to the isolation and therapeutic application of the unmodified vaPS.

### BACKGROUND OF THE DISCLOSURE

Regenerative cell therapy, which refers to the therapeutic application of stem cells to repair diseased or injured tissue, has received increasing attention from basic scientists, clinicians, and the public. Stem cells hold significant promise for tissue regeneration due to their innate ability to provide a renewable supply of cells that can form multiple cell types, whole tissue structures, and even organs. Stem cells are present in the human body at all stages of life from the earliest times of an embryo through adulthood and senescence.

Pluripotent stem cells are cells that have the capacity to self-renew by dividing and to develop into the three primary germ cell layers of the early embryo and therefore into all cells of the adult body, but not extra-embryonic tissues such as the placenta. Embryonic stem cells and induced pluripotent stem cells are primarily considered to be pluripotent stem cells

The current believe is that adult stem cells would be committed to becoming a cell from their tissue of origin, but might form other cell types as well. Some people call these cells tissue-specific stem cells. They have the broad ability to differentiate into cell types present in the respective organ, they reside in.” It contrasts with the definition of the term “multipotent mesenchymal stromal cells (MSCs)”, which is defined as “being adherent to plastic, expressing the surface markers CD73, CD90 and CD105, and having the ability to differentiate into osteoblasts, adipocytes and chondrocytes.”

However, it has been reported that cells can be isolated from bone marrow and vessel walls of adults that have the capacity to differentiate (upon stimulation, but without genetic reprogramming) into many more cell types than osteoblasts, adipocytes, and chondrocytes. Consequently, a recent study defined microvascular pericytes with the ability to produce somatic cells representative for the three primitive germ layers (ectoderm, mesoderm, and endoderm) as pluripotent adult stem cells, which is in contrast with the definitions above.

In fact, the situation is even much more complicated considering that several other surface markers of MSCs next to CD73, CD90, and CD105 were described, including (in alphabetical order) CD49f, CD146, CD200, CD271, CD349, GD2, MSCA-1, PODXL, Sox11, SSEA-3, SSEA-4, Stro-1, Stro-4, SUSD2, TM4SF1, and 3G5. This list was established based on reports of cells isolated from many different tissues, including (in alphabetical order) adipose tissue, amnion, bone marrow, decidua parietalis, dental pulp, dermis, endometrium, periodontal ligament, placenta, umbilical cord, and umbilical cord blood. However, not each of these surface markers was identified on MSCs isolated from each of the tissues listed above. Some of these markers were only found after cultivating cells for up to 100 days.

In this disclosure, the following terminology will be used: (i) A certain cell type can be isolated from different organs in the adult body (i.e., adipose tissue, heart, skin, bone marrow, or skeletal muscle) that can differentiate into ectoderm, mesoderm, and endoderm, providing significant support for the existence of a certain universal type of small, ubiquitously distributed, vascular-associated, pluripotent stem cell in the adult body (vaPS cells). (ii) These vaPS cells fundamentally differ from embryonic stem cells and from iPS cells in that the latter possess the necessary genetic guidance that makes them intrinsically pluripotent. In contrast, vaPS cells do not have this intrinsic genetic guidance. Nevertheless, they are able to differentiate into somatic cells of all three lineages under guidance of the microenvironment they are located in, independent from the original tissue or organ that they are derived from. (iii) As vaPS cells are contained in adipose-derived regenerative cells (ADRCs), the latter are able to form any somatic cell lineage guided by the respective tissue or organ they are applied to without the need for prior genetic modification. (iv) A cellular preparation that results from culturing fresh, unmodified ADRCs is called adipose-derived stem cells (ADSCs).

It has been proposed that pericytes would be the ancestors of perivascular MSCs, which would be in contrast to the concept of vaPS cells, as outlined in this paper. However, pericytes are fully differentiated cells that already have a terminal, differentiated purpose in life, namely the formation of capillaries together with endothelial cells. Two recent findings challenge the concept that pericytes would be the ancestors of perivascular MSCs: (i) culturing human ADSCs in a specific pericyte medium can induce pericyte-like differentiation of the ADSCs; and (ii) neuron-glial antigen 2 (NG2), which has long been associated with pericytes, was recently identified as a consistent surface marker of long-living human cord blood mesenchymal stem cells (LL-cbMSCs) that were fully characterized according to ISCT, and, to a lesser degree, of human bone marrow mesenchymal stem cells (vaPS cells were not investigated in this study). NG2 was also identified in extracellular vesicles produced by LL-cbMSCs. These data support the hypothesis that at least a subset of vaPS cells is also immunopositive for NG2 and, thus, NG2 is expressed by more cells than just by pericytes.

The reason why both vaPS cells and pericytes express NG2 may be explained by the fact that both cells must be in close contact to the (abluminal side of the) endothelial basal lamina. Specifically, vaPS cells are able travel to their destination via adjacent tissue and the blood stream upon activation, and the roles of pericytes in forming the typical capillary structure together with endothelial cells and vessel regulation require that they are located close to the endothelial basal lamina. This may be achieved by expression of NG2, as NG2 binds to Type VI collagen through the central nonglobular domain of its core protein, and Type VI collagen anchors endothelial basement membranes by interacting with Type IV collagen.

Therefore, there is the need to identify and isolate the vaPS that can be readily used in stem cell therapies.

### SUMMARY OF THE DISCLOSURE

In one aspect of this disclosure, a composition comprising isolated vascular-associated naturally pluripotent stem cells (vaPS) is disclosed. The vaPS are capable of differentiating into somatic cells of all three germ layers under guidance of the respective microenvironment.

In another aspect of this disclosure, a method of isolating small ubiquitously distributed vascular associated naturally pluripotent stem cells (vaPS) is disclosed. The method comprises: (a) obtaining a vascular tissue from a mammal, and (b) isolating cells expressing vaPS markers.

In another aspect of this disclosure, a therapeutic composition is disclosed, wherein the composition comprises isolated vascular-associated naturally pluripotent stem cells in a pharmaceutically acceptable carrier, wherein said vaPS is capable of differentiating into somatic cells of all three germ layers under the guidance of the respective microenvironment after the therapeutic composition is introduced into a mammal.

In yet another aspect of this disclosure, a method of treating a defect in a mammalian subject is disclosed. The method comprises: a) isolating unmodified autologous vascular-associated pluripotent stem cells (UA-vaPS) from the mammalian subject, and b) introducing the UA-vaPS into the mammalian subject at or near the defect.

In one embodiment, the isolated vaPS are isolated from vascular tissues. In one embodiment, the isolated vaPS are isolated from adipose tissues.

In one embodiment, the vaPS markers include at least one of 3G5+, NG2+, Nestin+, CD29+, CD49e+, SSEA4+, Oct4+, Nanog+, CXCR4+, CD34−, CD133−, CD144—, CD45−, CD11−, CD14−, CD68−.

In one embodiment, the UA-vaPS are not cultured or expanded in vitro prior to introducing into the mammalian subject to treat the defect.

In one embodiment, the defect includes but is not limited to: tendon defects, cartilage defects, chronic, recalcitrant low back pain caused by lumbosacral facet syndrome, avascular necrosis of femoral head, wounds, scar tissues, and hair loss.

As used herein, “vaPS”, or “vascular-associated pluripotent stem cells”, refers to stem cells obtained from different organs in the adult body (i.e., adipose tissue, heart, skin, bone marrow, or skeletal muscle) that can differentiate into ectoderm, mesoderm, and endoderm. In one embodiment, the vaPS are obtained from adipose tissue. In another embodiment, the vaPS are unmodified.

As used herein, “unmodified” means the cells have not been artificially manipulated.

As used herein, “adipose-derived regenerative cells” refers to cells obtained from adipose tissues, without being cultured, that are able to form any somatic cell lineage guided by the respective tissue or organ they are applied to without the need for prior genetic modification.

As used herein, “adipose-derived stem cells” refers to mesenchymal stem cells obtained from adipose tissues, adherent on plastic culture flask, can be expanded in vitro and have the capacity to differentiate into multiple cell linages.

As used herein, “vascular tissue” refers to a tissue having blood vessels and/or lymphatic vessels.

As used herein, “adipose tissue” refers to body fat that is a loose connective tissue composed mostly adipocytes, stromal vascular fraction of cells, as well as immune cells. In humans, adipose tissue is located at beneath the skin, around internal organs, in bone marrow, intermuscular, and in the breast. Adipose tissue also contains many small blood vessels.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| ADRC | Adipose-derived regenerative cells |
| ADSC | Adipose-derived stem cells |
| GFP | Green fluorescent protein |
| HVJ | Hemagglutinating virus of Japan |
| iPS | Induced pluripotent stem cells |
| NG2 | Neuron-glial antigen 2 |
| SCID | Severe combined immunodeficient |
| SFM | Serum-free medium |
| SMA | smooth muscle antigen |
| vaPS cells | Vascular-associated, pluripotent stem cells |

### BRIEF DESCRIPTION OF THE DRAWINGS

FIG. 1. Microvessel-associated cells (microvessel isolated from a rat brain) represent a universal resource within the entire body. (a) Scanning electron microscopic image of a microvessel. (b) Phase contrast image of a microvessel as used for further analysis. The scale bar represents 5 μm in (1) and 40 μm in (b).

FIG. 2. Scanning electron microscopic image of cells that were freshly isolated from human abdominal adipose tissue (reprinted from the permission from the sportärztezeitung). Abbreviations: P, progenitor cells; S, small cells; D, dying cells; L, lymphocytes; E, exosomes. The white arrows point to actin filaments, and the white arrowhead to a micro-channel between two cells. The scale bar represents 10 μm (20 μm in the high-power inset).

FIG. 3. Immunofluorescence detection of neuron-glial antigen 2 (NG2) (red in (a,c,d); arrows), smooth muscle antigen (SMA) (green in (b-d) and laminin (purple in (d) in the wall of a small human arteriole (cell nuclei in blue). The scale bar represents 20 μm in (a, b) and 10 μm in (c, d).

FIG. 4. Immunofluorescence detection of NG2 (a), Nestin (b), CD29 (c), CD44 (d), CD146 (e), smooth muscle antigen (SMA) (f), CD73 (g), and CD105 (h) in cells that were freshly isolated from human adipose tissue. Note the very small cytosol of the cells that were immunopositive for NG2, Nestin, and CD29 (a-c). The scale bar represents 10 μm (a-h).

FIG. 5. Schematic illustration of the hypothesized location of the vaPS cells.

FIG. 6. Expression of surface markers of vaPS cells in their primary, secondary and tertiary niche.

FIG. 7. Change in the morphology of rat adipose-derived stem cells cultured first in fetal bovine serum (FBS) for 14 days (a), then in serum-free medium (SFM) for 24 h (b), and then again in FBS for one week (c). Afterwards, transfer to SFM again induced a spheroid-like appearance (d). The arrows indicate the consecutive order of the panels. The scale bar represents 100 μm.

FIG. 8. Aggregation of human adipose-derived stem cells, leading to the formation of spheroids that resemble the appearance of embryoid bodies formed by embryonic stem cells (reprinted from Winnier et al. with permission from PLOS). The scale bar represents 50 μm in the right panel.

FIG. 9. Immunofluorescence detection of CD11b (a), CD14 (b), CD31 (c), CD34 (d), CD45 (e), and HLA-DR (f) on the surface of human adipose-derived stem cells that were cultured for four days in serum-free media. The scale bar represents 60 μm in (a, d, e), 20 μm in (b), 40 μm in (c), and 30 μm in (f).

FIG. 10. Immunofluorescence detection of CD44 (a), CD73 (b), CD90 (c), CD105 (d), Nestin (e), and smooth muscle antigen (SMA) (f) on the surface of human adipose-derived stem cells that were cultured for four days in serum-free media. The scale bar represents 16 μm in (a,c), 12 μm in (b,d,f), and 20 μm in (e).

FIG. 11. Immunofluorescence detection of Oct4 (a) and neuron-glial antigen 2 (NG2) (b) in cells in spheroids that were created by unmodified human adipose-derived stem cells that were cultured for four days in serum-free media. The scale bar represents 20 μm.

FIG. 12. Clonal expansion of a single human vaPS cell (i.e., a single human ADSC) for five days in fetal bovine serum (FBS), followed by multilineage differentiation induced by culturing the cells for three weeks in the respective induction media (protocols are provided in [10]). (a-d) Culturing of cells in FBS for 6 h (a), 20 h (b), 48 h (c), and five days (d). (e-h) Induction of adipogenesis (mesoderm) (e), osteogenesis (mesoderm) (f), hepatogenesis (endoderm) (g), and neurogenesis (ectoderm) (h), confirmed by oil red O staining (e) and alizarin red S staining (f) as well as immunofluorescence detection of albumin (specific for hepatocytes) (g) and microtubule-associated protein 2 (MAP-2) (specific for neurons) (h). The scale bar represents 80 μm in (a-d), 100 μm in (e,f), and 40 μm in (g,h).

FIG. 13. Induction of adipogenesis (mesoderm; confirmed by oil red O staining) (a,e,i,m,q), osteogenesis (mesoderm; confirmed by alizarin red S staining) (b,f,j,n,r), hepatogenesis (endoderm; confirmed by immunofluorescent detection of albumin) (c,g,k,o,s), and neurogenesis (ectoderm; confirmed by immunofluorescent detection of MAP-2) (d,h,l,p,t) by culturing rat vaPS cells obtained from adipose tissue (a-d), heart (e-h), skin (i-1), bone marrow (m-p), and skeletal muscle (q-t) for three weeks in the respective induction media (previously unpublished figure). The scale bar represents 100 μm in (a,e,m,q), 120 μm in (b), 50 μm in (c,d,f-h,j,n,r), 20 μm in (i), 70 μm in (k), 30 μm in (1,p), 25 μm in (o), and 60 μm in (s,t).

FIG. 14. Induction of adipogenesis (mesoderm; confirmed by oil red O staining) (a), osteogenesis (mesoderm; confirmed by alizarin red S staining) (b), hepatogenesis (endoderm; confirmed by immunofluorescent detection of albumin) (c) and neurogenesis (ectoderm; confirmed by immunofluorescent detection of MAP-2) (d) by culturing human adipose-derived stem cells (ADSCs) for three weeks in the respective induction media (previously unpublished figure). No induction of three germ layer differentiation was observed by culturing human ADSCs in non-inductive control media (e-h). The scale bar represents 100 μm in (a,b,e,f) and 40 μm in (c,d,g,h).

FIG. 15. Staining of cells that are immunopositive for troponin T, as well as cytosol of human ADSCs. (a) Immunofluorescence detection of troponin T in the cytoplasm of a rat cardiomyocyte (red signal) (adapted from Metzele et al. with permission from John Wiley & Sons—Books) Human adipose-derived stem cells that were labeled with green fluorescent protein (GFP) (green signal) (previously unpublished figure). The arrow points to a GFP-positive cell that additionally was immunopositive for MF20 (resulting in a yellowish signal). The scale bar represents 20 μm in (a) and 40 μm in (b).

FIG. 16. Cell-cell contact between a rat cardiomyocyte (expressing troponin T; red signal) and a cell expressing both green fluorescent protein and troponin T (yellowish signal), representing a human adipose-derived stem cell at the latest stage of culturing (adapted from Metzele et al. with permission from JohnWiley & Sons—Books). The inset shows the contact between the cells at higher magnification. The scale bar represents 15 μm (5 μm in the inset).

FIG. 17. Phase-contrast images (a,b), fluorescence images (c,d) and merged images (e,f) of heart sections prepared four weeks after experimental induction of myocardial infarction in severe combined immunodeficient (SCID) mice and injection of human adipose-derived stem cells (ADSCs) (a,c,e) or human adipose-derived regenerative cells (ADRCs) (b,d,f) into the peri-infarct region (adapted from [9] with permission from Oxford University Press). Cell nuclei were labeled with DAPI (blue); nuclei of human cells were labeled with an antibody against human lamin A/C (green); and gap junctions were labeled with an antibody against connexin 43 (red). Yellow arrows indicate detection of connexin 43 in gap junctions that could not be attributed to cell-cell contacts between mouse cells but most probably represented cell-cell contacts between mouse cardiomyocytes and descendants of injected human cells. The scale bar represents 15 μm in (a) and 10 μm in (b).

FIG. 18. Representative photomicrographs of a paraffin-embedded, 5-μm-thick tissue section of a post-mortem heart from a pig, taken from the left ventricular border zone of myocardial infarction ten weeks after experimental occlusion of the left anterior descending (LAD) artery for three hours, followed by the delivery of eGFP-labeled autologous adipose-derived stem cells into the balloon-blocked LAD vein (matching the initial LAD occlusion site) at four weeks after occlusion of the LAD (previously unpublished figure). The section was stained with DAPI (blue) and processed for immunofluorescence detection of GFP (green), connexin 43 (Cx43) (red), and troponin (yellow). The circles indicate regions where most of the cell nuclei were immunopositive for GFP, and the arrow a GFP-positive cell nucleus inside a cardiomyocyte. The scale bar represents 25 μm in the merged panel, and 50 μm in the individual panels.

FIG. 19. Time-lapse video microscopy of human adipose-derived stem cells (ADSCs) that were co-cultured with MDAMB-231 cells labeled with green fluorescent protein (GFP) (green signal in (h-t)) (previously unpublished figure). One of the ADSCs shown in this figure was labeled with a red quantum dot (red signal in all panels; the corresponding cell body is marked by a white arrow in all panels). In (f), this ADSC formed a contact with another ADSC (yellow arrows in (b-i); the cell-cell contact is indicated in (f). However, the quantum dot was not exchanged between the cells. In (o) the same ADSC formed a contact with a MDA-MB-231 cell (green arrows in (h-t)); the cell-cell contact is indicated in (o) and the quantum dot was transferred from the ADSC to the MDA-MB-231 cell. The high-power inset in (o) shows that the quantum dot left the ADSC through a microchannel that was formed by the ADSC itself, which demonstrates active participation of the ADSC in exchange of cellular components between the cells. The time interval between the frames was 40 min each. The scale bar represents 50 μm in (a-t), and 25 in the inset in (o).

FIG. 20. Time-lapse video microscopy of human adipose-derived stem cells (ADSCs) that were co-cultured with MDA-MB-231 cells labeled with green fluorescent protein (GFP). Several of the ADSCs shown in this figure were labeled with red quantum dots. A quantum dot in the cell body of one of the ADSCs is marked by a red arrow in (b-h); the corresponding cell is marked by a blue arrow in all panels. The white arrow in all panels point to an exosome. The yellow arrow in (j) indicates the event of pinocytosis of the exosome by the ADSC that is marked by the blue arrow. The time interval between the frames was 20 min each. The scale bar in (t) represents 50 μm.

FIG. 21. Time-lapse video microscopy of human adipose-derived stem cells (ADSCs) that were co-cultured with MDAMB-231 cells labeled with green fluorescent protein (GFP) (green signal in all panels; the red signal in the panels came from red quantum dots that were exchanged between the ADSCs and the MDA-MB-231 cells (c.f. FIG. 19) (previously unpublished figure). The white arrow in all panels indicates an ADSC that underwent apoptosis (named “white cell”; the disintegration of the cell body of the white cell is marked in (j) by an asterisk). The red, yellow, green and blue arrows indicate four other cells (named “red cell”, “yellow cell”, “green cell”, and “blue cell”) that migrated towards the white cell during the three hours before disintegration of the cell body of the white cell (a-i). The red cell was an ADSC whereas the yellow, green and blue cells were MDA-MB-231 cells. During the first 90 min after disintegration of the cell body of the white cell the other cells took up apoptotic bodies of the white cell by means of pinocytosis (indicated by a “P” in the respective color in (j,k,m,n)). The easiest way to recognize these pinocytosis events is to sequentially follow each cell through the panels (a-t). The time interval between the frames was 20 min each. The scale bar in (t) represents 50 μm.

FIG. 22. Homeostasis between dying cells (gray) and stem cells (orange) in tissue under healthy conditions (a), the situation under pathological conditions as well as during aging (b), and after stem cell therapy (c) (previously unpublished figure). Under healthy conditions, tissue homeostasis is maintained between dying cells and replacing stem cells. In contrast, tissue homeostasis is disrupted under pathological conditions as well as during aging as more cells are dying than being replaced by stem cells. Application of a concentrated stem cell preparation can re-establish tissue homeostasis.

FIG. 23. In vivo bioluminescence imaging over time of 5×10⁵ human adipose-derived stem cells (ADSCs) that were intramyocardially delivered into SCID mice after experimental induction of myocardial infarction by permanent ligation of the left anterior descending coronary artery (adapted from Bai et al. with permission from Springer Nature). The ADSCs were transfected with a lentiviral vector expressing green fluorescent protein and luciferase. Strong bioluminescence signals were found over the heart region at all investigated time points (arrows), indicating the long-term survival of delivered ADSCs in injured hearts. The investigated time points are provided below the panels.

FIG. 24. In vivo bioluminescence imaging over time of human adipose-derived stem cells (ADSCs) that were delivered together with crosslinked collagen into a subcutaneous location of SCID mice. The ADSCs were transfected with a lentiviral vector expressing green fluorescent protein and luciferase. Strong bioluminescence signals were found at the injection site up to 6 month (arrows), indicating the long-term survival of delivered ADSCs in subcutaneous tissue. (h) Immunofluorescence detection of von Willebrand factor (vWF; red signal) and Lamin A/C (green signal) as an indicator of human cells in a SCID mouse, forming a blood vessel four weeks after application of the human ADSCs into a subcutaneous wound (counterstaining with DAPI; blue signal). The scale bar represents 20 μm in (h).

FIG. 25. Tendon defects treated with ADRCs. (a,b) Original (asterisk) and newly formed (arrows) tendon tissue in a section of a biopsy from a human supraspinatus tendon that was taken ten weeks post-treatment of a traumatic sPTRCT using UA-ADRCs (reprinted from with permission from the sportarztezeitung) (a): immunohistochemical detection of type I collagen; (b): polarization microscopic image of the section shown in (a). Note the organized, slightly undulating type I collagen and the high cell density in the newly formed tendon tissue which does not resemble scar tissue (details in Alt et al., 2021). (c) Immunohistochemical detection of tenomodulin in a section of the same biopsy (section adjacent to the one shown in a) (Panels (c-e) reprinted from Alt et al., 2021, with permission from Baishideng Publishing Group). The insets show the position of Panels (d) and (e) (as well as the position of Panels (a,b) in the adjacent section). (d) Position of a spot with very high density of cells and microvessels (yellow arrows, intracellular immunolabeling for tenomodulin; yellow asterisks, extracellular immunolabeling for tenomodulin). (e) Position of degenerative tendon tissue with formation of microvessels (black arrows, intracellular immunolabeling for tenomodulin in cells inside microvessels). The scale bar represents 100 μm in (a,b), 630 μm in (c), and 50 μm in (d,e).

FIG. 26. Example of successful application of unmodified, autologous, adipose-derived regenerative cells (UA-AORCs) for treating cartilage defects (reprinted from Alt et al., 2020, with permission from MDPI). The panels show arthroscopic views of the right (a-c) and the left (d) knee of a male, 51-year-old subject who presented with recurring and increasing pain in both knee joints during walking and other activities. (a) Third-degree damage to the right tibia 1 plateau (white asterisk) after a tibial chondrocyte transplantation that had been performed three years previously, as well as considerable osteoarthritic damage of the femoral cartilage (black asterisk). (b) Situation of the right knee after arthroscopic removal of the failed chondrocyte transplant (white asterisk) as well as ‘mushy’ and damaged cartilage structure on the femoral condyles before it was removed (black asterisk). (c) Situation of the right knee one year after arthroscopic removal of damaged cartilage and a single application of UA-ADRCs isolated from 100 mL of lipoaspirate, showing complete healing of the tibial defect (white asterisk) and of the femoral parts, with formation of new whitish cartilage that shows a sharp demarcation border to the existing old and more yellowish cartilage (arrows). (d) Situation of the left knee one year after performing a standard procedure without application of UA-ADRCs (i.e., arthroscopic removal of damaged cartilage and drilling of small holes into the bone), showing a somewhat uneven, overshooting fibroblastic scar formation (black asterisk) without a sharp demarcation border to the original cartilage (arrows). Abbreviations: R, right; L, left; BL, baseline; M12, twelve months after baseline.

FIG. 27. Other examples of successful application of unmodified, autologous, adipose-derived regenerative cells (UAADRCs) for treating cartilage defects (previously unpublished figure). The panels show arthroscopic views of the knees of two subjects (Subject 1 (male, 45 years old): (a,b); Subject 2 (male, 55 years old): (c,d) before (a,c) and one year after (b,d) arthroscopic removal of damaged cartilage (asterisks in (a,c)) and application of UA-ADRCs isolated from 50 mL of lipoaspirate. The arrows in (b,d) indicate the sharp demarcation border between the newly formed and the original cartilage. The white arrow in (a) points to the arthroscopic instrument that was used to remove damaged cartilage.

FIG. 28. Representative photomicrographs of 5-μm-thick histologic sections (stained with toluidine blue (a,b) or hematoxylin and eosin stain (c,d)) of the small tissue samples taken during arthroscopic inspection of the knees of the subject represented in FIG. 26 at one year after arthroscopic removal of damaged cartilage and a single application of unmodified, autologous, adipose-derived regenerative cells (UA-ADRCs) (right knee) (a,c) or after performing a standard procedure without application of UA-ADRCs (i.e., arthroscopic removal of damaged cartilage and drilling of small holes into the bone) (left knee) (b,d), respectively (reprinted from with permission from MDPI). The dotted lines in (a) (knee treated with UA-ADRCs) indicate a zonal organization of the newly formed cartilage with differently shaped chondrocytes in a superficial (SL), middle (ML) and deep layer (DL). The arrows in (b) (knee treated with a standard procedure without application of UA-ADRCs) point to scattered cells within newly formed amorphous fibrocartilage. The arrows in (c) (knee treated with UA-ADRCs) indicate typical chondrocytes with a small nucleus and a hollow space around in the directly built contact zone between the newly formed cartilage and bone. In contrast, the arrows in (d) (treatment without UA-ADRCs) point to an infiltration with inflammatory cells in the contact zone between a newly formed fibro-cartilage and bone. The arrowheads in (d) indicate three small blood vessels. The scale bar represents 100 μm in (a-d).

FIG. 29. Representative photomicrographs of 5-μm-thick histologic sections (stained with hematoxylin and eosin stain) of the small tissue sample taken during arthroscopic inspection of the right knee of the subject represented in FIG. 26 at one year after arthroscopic removal of damaged cartilage and a single application of the subject's own, unmodified, autologous, adipose-derived, regenerative cells (previously unpublished figure). The panels (b,d) were generated with polarized light microscopy and converted to grayscale. The red lines in (b,d) indicate the orientation of the collagen fiber bundles within the newly formed cartilage (more vertically in in the deep and middle layers, and more horizontally in the superficial layer). The asterisk in (c) indicates a region of contact between the original cartilage and bone, demonstrating that the tissue sample was indeed taken from the newly formed cartilage, including the original border zone between bone at the bottom and newly formed cartilage on top (dotted line). The scale bar represents 100 μm in (a-d).

FIG. 30. Results of treatment of low back pain caused by Lumbosacral Facet Syndrome. (a-c) Schematic of the lumbar vertebrae L3 to LS and the upper edge of the os sacrum of a human spine from dorsal (a) and lateral (b,c) (previously unpublished figure). The red arrows indicate the zygapophyseal (facet) joints between L3 and L4 (L3/L4), between L4 and LS (L4/LS) and between LS and the os sacrum (LS/S1) with joint capsule (on the left side in (a,b)) as well as without joint capsule (on the right side in (a,c)). The green arrows in (b,c) indicate the intervertebral disc between L3 and L4, and the yellow arrows the intervertebral disc between LA and LS. (d-f) MRI scans of the lumbar spine (from lateral) of three former professional, internationally highly successful ski racers. The green arrows indicate normal intervertebral disc structure between L3 and LA, whereas the yellow arrows show that, in all three athletes, there was a reduction in the height of the intervertebral disc (and, thus, the intervertebral space) between L4 and LS. The asterisk in (f) points to a bone marrow edema in the vertebral body of LS, and he black arrow in (f) to an upper plate collapse of LS. All three athletes were treated with unmodified autologous adipose-derived regenerative cells. The cells were injected at the site of the right and left facet joints between L4 and LS as well as between LS and SI within a single procedure of harvesting and injection that lasted about two hours. This treatment resulted in significant and long-lasting (now more than three years) pain reduction that enabled one of these athletes to very successfully return to competitive sports.

FIG. 31. Individual pain scores on a VAS scale. (a) Connected individual VAS scores of n=39 subjects with chronic, recalcitrant low back pain caused by lumbosacral facet syndrome before (red dots) and after (green dots) treatment with UA-ADRCs isolated from 100 mL of lipoaspirate each (the follow-up data ranged between one and more than three years) (previously unpublished figure). The subjects were listed consecutively according to the date of treatment. (b) Mean and standard deviation of the individual improvement of the VAS score of the subjects shown in (a) as a function of the VAS score at baseline (i.e., before treatment). The results of linear regression analysis (regression line with 95% confidence interval) are also indicated (r2=0.22; p=0.003).

FIG. 32. Results of using UA-ADRCs in guided bone regeneration. (a,b) Representative photomicrographs of 3-μm-thick paraffin sections stained with hematoxylin and eosin stain of biopsies that were collected at 34 weeks after performing guided bone regeneration (in the framework of a bilateral external sinus lift procedure as well as a bilateral lateral alveolar ridge augmentation in a 79-year-old subject who presented with a partly failing maxillary dentition) using a combination of approximately 50×10⁶ unmodified, autologous, adipose-derived regenerative cells (UA-ADRCs), Fraction 2 of plasma rich in growth factors (PRGF-2) and an osteoinductive scaffold (OIS) (right side) (a) or a combination of PRGF-2 and the same OIS alone (left side) (b) (reprinted from Solakoglu et al. with permission from Baishideng Publishing Group). Abbreviations: A, adipocytes; B, newly formed bone; S, scaffold; V, veins. The scale bar represents 100 μm in (a,b). (c,d) MRI scans of the left hip of a 41-year-old male subject suffering from avascular necrosis of the femoral head (arrows in (c,d)) at baseline (c) and at six months after two applications of UA-ADRCs.

FIG. 33. Examples of successful application of unmodified autologous adipose-derived regenerative cells (UA-ADRCs) for treating chronic wounds that did not heal for years (previously unpublished figure). (a-d) Female, 76-year-old subject; chronic wound over the medial malleolus; single application of UA-ADRCs isolated from 30 mL of lipoaspirate. (e-h) Male, 82-year-old subject; chronic wound on the lower leg caused by a phosphorus bomb during World War II; single application of UA-ADRCs isolated from 30 mL of lipoaspirate. (i-1) Female, 84-year-old subject, chronic wound on the lower leg; single application of UA-ADRCs isolated from 30 mL of lipoaspirate. Abbreviations: BL, baseline; D9, nine days after application of UA-ADRCs; MI/M2/M2.5/M3/M5/M7, one/two/two and a half/three/five/seven months after application of UA-ADRCs.

FIG. 34. Examples of successful application of unmodified, autologous, adipose-derived regenerative cells (UA-ADRCs) for reducing scar tissue (previously unpublished figure). (a,b) Male, 48-year-old subject; scar tissue formation on the upper arm after an injury caused by a car accident; single application of UA-ADRCs isolated from 100 mL of lipoaspirate. (c,d) Male, 50-year-old subject; scar tissue formation (arrows) in the face after an acid attack; three treatments with UA-ADRCs isolated from 100 mL of lipoaspirate on days 1, 90, and 180. Abbreviations: BL, baseline; M1/M12, one/twelve months after application of UA-ADRCs.

FIG. 35. Examples of successful application of unmodified, autologous, adipose-derived regenerative cells (UA-ADRCs) for treating hair loss (previously unpublished figure). (a,b) 46-year-old female subject; sparse hair; single application of UA-ADRCs isolated from 100 mL of lipoaspirate. (c-f) 23-year-old female subject suffering from atopic dermatitis and alopecia totalis for three years without spontaneous improvement; three treatments with UA-ADRCs isolated from 100 mL of lipoaspirate each at baseline (c) and at three and six month after the first treatment. Results are shown at one (M1; (d)), three (M3; (e)) and seven (M7; (f)) month after the first treatment.

### DETAILED DESCRIPTION

The disclosure provides novel discovery and use of vascular-associated pluripotent stem cell that can be used in stem cells therapies without any modification.

As defined above, vaPS cells are neither pluripotent stem cells as defined by CIRM nor MSCs, as defined by ICST and ISSCR. Specifically, vaPS cells do not yet express CD73, CD90, and CD105. This disclosure discovers the distinction between vaPS and MSCs that have not been described before. Furthermore, it should be mentioned that other authors described very small cells with reduced metabolic activity and pluripotent potential in the adult body in the past. However, these cells were not described in the literature as ubiquitously distributed and vascular-associated.

Stem cells are ubiquitously present in tissue that contains blood vessels. Blood vessels are the initial structures to be formed when a new organ is developing in an embryo. The presence of vaPS cells in the vascular location allows equal distribution of stem cells with pluripotent capacity (except for forming placental tissue) throughout the body. These cells are assumed to serve as a repertoire for renewal of the respective tissue and organs for the rest of the life of the individual.

For example, a certain number of stem cells per gram tissue can be isolated from rat brain tissue. However, when preparing a microvascular preparation of rat brain tissue in which only microvessels were remaining and the rest of the brain tissue was discarded—we found that the resulting number of stem cells per gram tissue increased by several potencies, indicating that the majority of stem cells are indeed located or associated with the vascular structure. Moreover, we were able to demonstrate that the same vaPS cells can be isolated from blood vessels independent of the organ or tissue they are derived from.

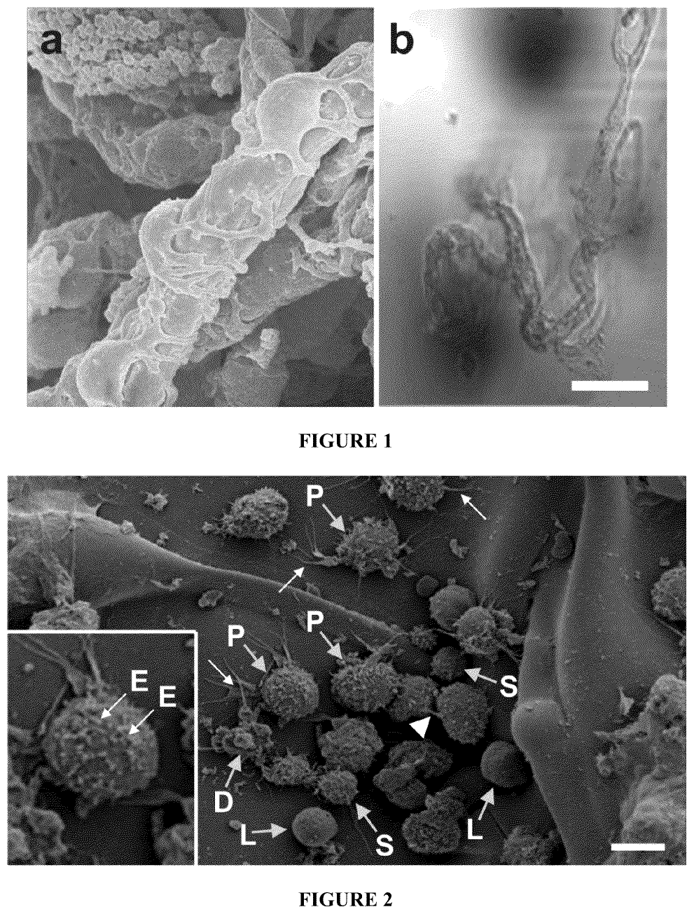

Our results demonstrate that vaPS cells can be isolated from all blood vessels, independent of the organ or tissue that they are derived from. This was demonstrated with cells derived from both human and animal tissue. Specifically, these vaPS cells were isolated from microvessels. FIG. 1 shows a microvessel preparation and a phase contrast image of such a microvessel from a rat brain.

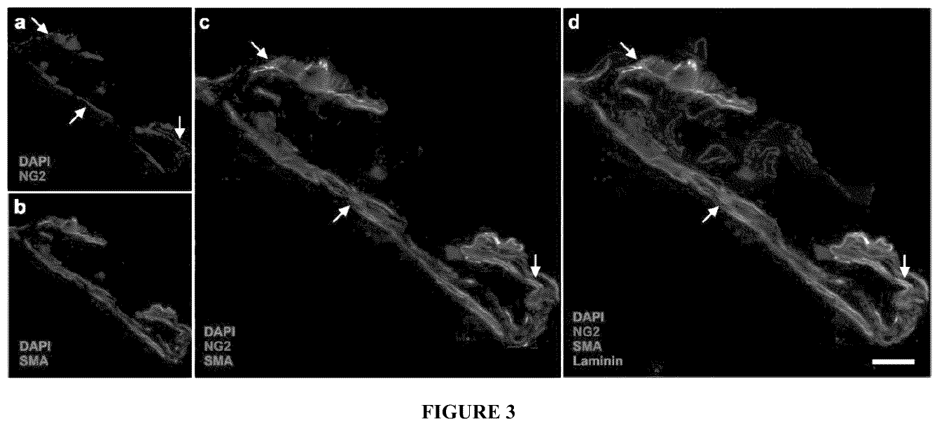

FIG. 2 shows a scanning electron microscopic image of cells that were freshly isolated from human abdominal adipose tissue by enzymatic release, and were imaged just minutes after isolation and plating. These cells (which represent the ADRCs) were not cultured but freshly isolated. The presence of a composition of different cells is found in this image. One can recognize larger cells that exhibit a rough surface structure (‘P’ in FIG. 2). These cells are progenitor cells that have already started to build actin filaments (white arrows in FIG. 2) in order to enhance their adherence to the extracellular matrix on which they were seeded. Besides this, some of these cells have started to communicate through microtubular structures (arrowhead in FIG. 2).

It should be mentioned that, in contrast to stem cells, progenitor cells are already on a pre-determined pathway to become a differentiated cell and have lost their ability to decide what they want to be “in life.” Accordingly, progenitor cells are typically determined to differentiate and develop into a lineage defined cell type. For example, in bone marrow, more than 99% of the cells are not stem cells, but primarily hematopoietic progenitor cells. Accordingly, progenitor cells have already started a pathway of lineage-committed differentiation. In the case of bone marrow-derived cells, these hematopoietic progenitor cells (often incorrectly labeled as stem cells) started to differentiate into future hematopoietic cells of the white, red, or platelet lineage. Pending on their progress in maturation in this differentiation process, these cells are no longer able to significantly revert their pathway of differentiation. At best, they are able to vary somewhat within the same germ layer of differentiation, but typically stay within the same lineage.

In addition, there are also lymphocytes present in the cellular preparation that was freshly isolated from human abdominal adipose tissue by enzymatic release (‘L’ in FIG. 2), as well as small cells (‘S’ in FIG. 2). These small cells show a relatively smooth surface and are limited yet in their ability to adhere to the extracellular matrix. They do not show exosomal structures on their surface as some of the larger cells do (′E′ in the high-power inset in FIG. 2). In addition, dying cells are found which are devolving into apoptotic bodies (‘D’ in FIG. 2).

Analyzing the individual cells shown in FIG. 2 in more detail revealed that approximately 20% of the cells were ‘naked’ and smooth on their surface, most likely representing white blood cells such as lymphocytes (‘L’ in FIG. 2). The content of the small assumed vaPS cells (‘S’ in FIG. 2) was about 10% of the cell population shown in FIG. 2. This demonstrates that, following enzymatic preparation, ADRCs of human abdominal adipose tissue are composed of vaPS cells, white blood cells, and larger cells, most likely progenitor cells. However, the composition between the different cellular elements varies with the tissue these cells are obtained from.

A more precise location of the cells in the vascular structures is revealed by multicolored immunohistochemistry of a small arteriole (FIG. 3). Cell nuclei (blue in FIG. 3) and smooth muscle antigen (SMA) (green in FIG. 3) define the structure of the arteriole. Furthermore, the location of laminin (purple in FIG. 3) correlates with the border between the endothelial basal lamina (representing the intima) and the media containing the smooth muscle cells (note that in this position also the internal elastic lamina is found). Of note, NG2 (red in FIG. 3) was found in close proximity to laminin.

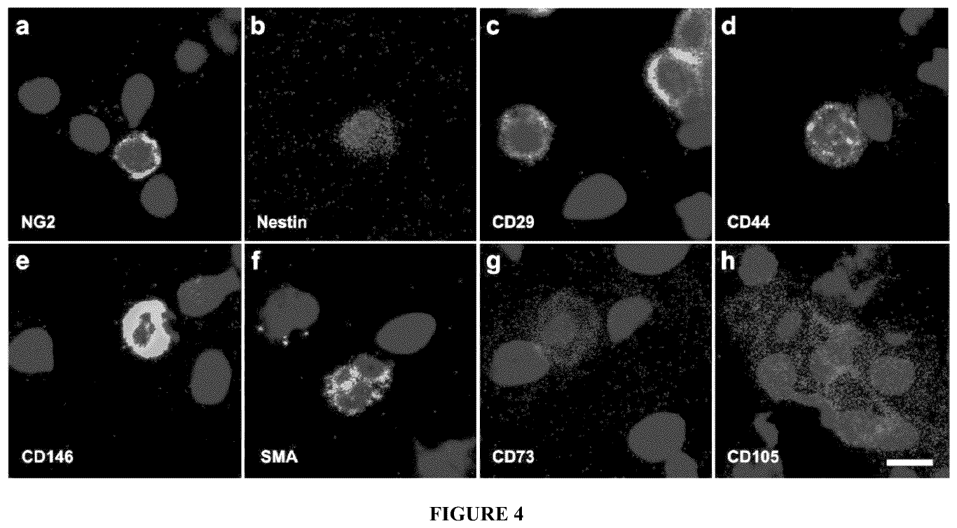

Using immunofluorescence we were able to detect NG2, Nestin, CD29, CD44, CD146, smooth muscle antigen (SMA), CD73, and CD105 in human cells that were freshly isolated from adipose tissue (FIG. 4). Of note, cells immunopositive for NG2, Nestin, and CD29 showed a very small cytosol (FIG. 4a-c).

Nestin is an early marker of neural stem/progenitor cells as well as of proliferative endothelial cells. These cells have a tiny cytosol compared to the nucleus and to other, more differentiated cells. Furthermore, cells immunopositive for CD29 (integrin β1) exhibit this kind of small cytosolic immunostaining (FIG. 4c). Together with CD49c (integrin α-5) CD29 forms integrin α5β1, the primary receptor for fibronectin. Most probably, the latter attaches the vaPS cells to the extracellular matrix within the vessel wall. This is supported by the fact that SPARC (secreted protein acidic and rich in cysteine; also known as osteonectin) can mobilize ADSCs through its effect on integrin α5β1, providing a functional basis for the regulation of the contribution of these cells to tissue and organ repair by SPARC. The latter is synthesized by several types of cells, including osteoblasts and odontoblasts, as well as endothelial cells and fibroblasts, but also macrophages, infiltrating leukocytes and cancer cells. Thus, SPARC may represent a key regulator in making vaPS cells a replacement source responsive to the signals of the surrounding tissue.

It is of note that SPARC is also expressed by ADSCs in vitro. Moreover, the SPARC-related modular calcium-binding protein 1 (SMOC1), a member of the SPARC family and serving as a regulator of osteoblast differentiation, was found in the secretome of bone marrow-derived MSCs. Thus, SPARC may play a pivotal role in both affecting the properties of vaPS cells in terms of proliferation and differentiation based on cues from the extracellular environment, as well as in paracrine activities of vaPS cells, impacting upon the activities of other cells in the local microenvironment.

Other staining and flow cytometric analyses showed that vaPS cells are additionally positive for Oct4, Sca1, and SSEA4.

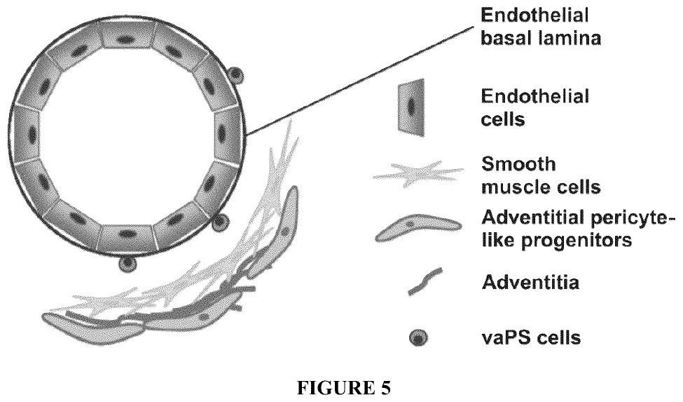

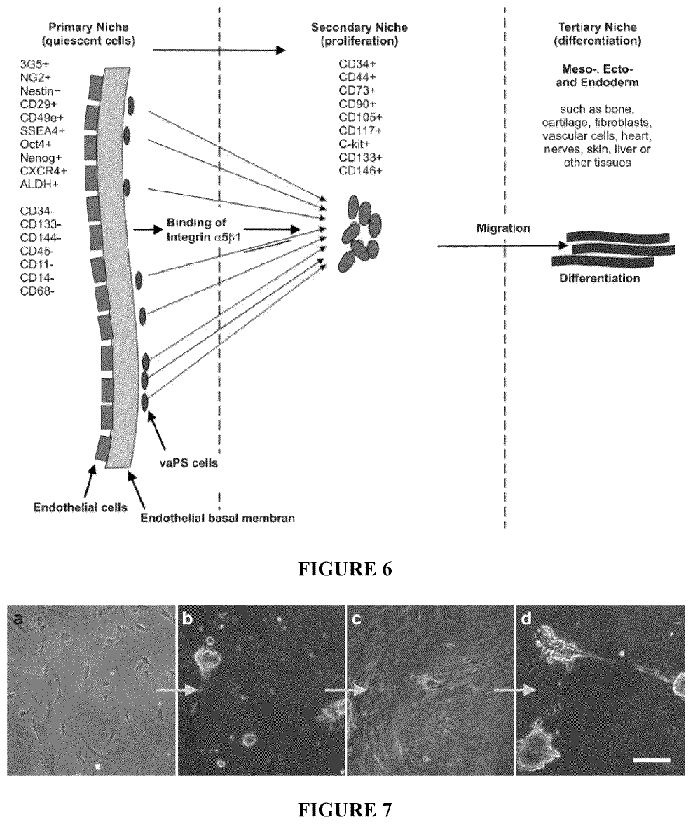

We therefore propose universal, vascular-associated stem cells through our findings. FIGS. 5 and 6 illustrate our hypothesis regarding the location and surface marker expression of vaPS cells. FIG. 5 shows the endothelial basal lamina in black and the endothelial cells on the luminal side of the endothelial basal lamina in blue. We hypothesize that the vaPS cells reside opposite to the endothelial cells on the abluminal side of the endothelial basal lamina, towards the smooth muscle cell layer and embedded within those. We are aware of the fact that this is in contrast to other descriptions in the literature that vascular-associated MSCs, presumably immunopositive for pericyte markers, would be located in the adventitia.

The vaPS cells are small, which allows them to migrate through tissue in order to help maintaining tissue homeostasis. When these cells leave their quiescent location (i.e., their primary niche, as depicted in FIG. 6), they start to migrate, followed by proliferation which is primarily under control of the Wnt signaling pathway, and finally differentiation. The location of proliferation may be called the secondary niche (FIG. 6), in contrast to the primary niche that is assigned to the vessel wall (FIGS. 5 and 6). In this regard, we hypothesize that markers such as CD44, CD73, CD90, and CD105—often believed to be indicative of stem cells—are only present in cells that have already left their primary niche and started to enter the next developmental phase in order to attain progenitor status. This hypothesis is supported by the fact that CD44, CD90, and CD105 are also expressed by fibroblasts that exhibit no plasticity at all. Finally, the cells can leave their secondary niche and differentiate in the tertiary niche (FIG. 6) into their final lineage. The tissue specific differentiation pathway is controlled by signaling from the cells' new microenvironment, including micro-RNA and transcription factors. Accordingly, throughout life, replacing cells exist that can be mobilized upon need for tissue renewal and repair.

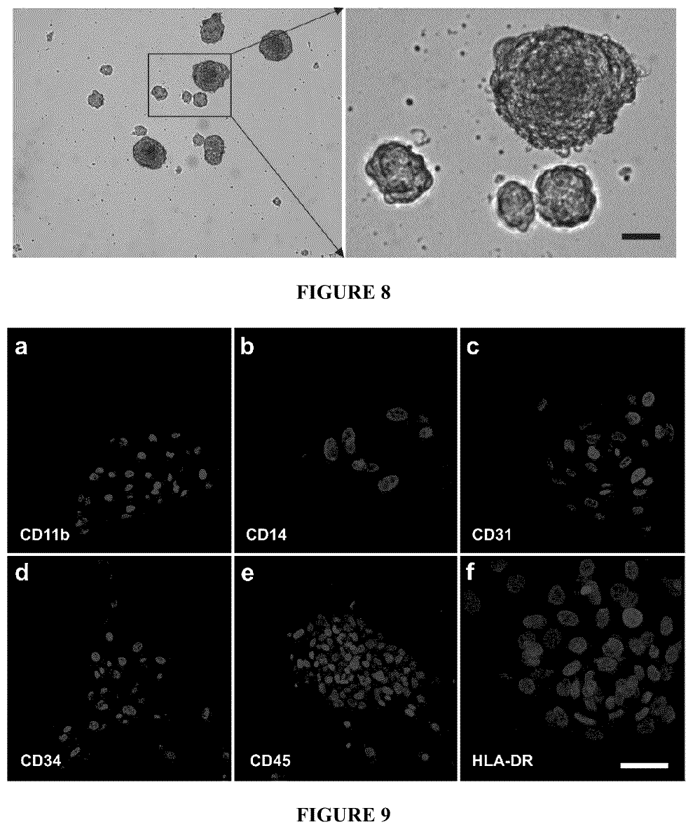

Surface markers of vaPS cells cultured in serum-free media can also be used to distinguish other cell types. When cultured in fetal bovine serum (FBS), ADSCs display a typical, spindle-shaped appearance (FIG. 7a,c). In contrast, they form spheroids when cultured in serum-free media [SFM] (FIG. 7b,d). After culturing ADSCs for seven days in SFM, we found spheroid bodies with a diameter of up to 250 μm (FIG. 8). The shape of these spheroid bodies resembled pretty much the shape of spheroid bodies formed by embryonic stem cells, known as embryoid bodies.

Immunofluorescence analysis of human ADSCs that were cultured for four days in SFM showed that these cells were immunonegative for CD11b (a marker of macrophages), CD14 (a marker of hematopoietic progenitor cells), CD31 (a marker of endothelial progenitor cells), CD34 (a marker of progenitor cells in general), CD45 (a pan-leukocyte marker), and HLA-DR (FIG. 9). Corresponding flow cytometric analysis revealed that the relative numbers of cells that were immunopositive for these surface markers were smaller than 1.5% (relative number of cells that were immunopositive for CD11b (CD11b+): 1.1%; CD14+: 0.6%; CD31+: <0.1%; CD34+: 0.4%; CD45+: 1.3% and HLA-DR: 0.2%). These results confirmed that, after culturing ADSCs for four days in SFM, macrophages, hematopoietic progenitor cells, endothelial progenitor cells, progenitor cells in general, cells expressing the pan-leukocyte marker CD45, and cells expressing HLA-DR were virtually absent in this cell culture. Therefore, culturing ADSCs in SFM is a powerful tool to discriminate between vaPS cells, progenitor cells, and differentiated cells. We hypothesize that the loss of CD34+ cells after culturing ADSCs for four days in SFM is a consequence of missing signaling from the normally surrounding microenvironment in the natural tissue.

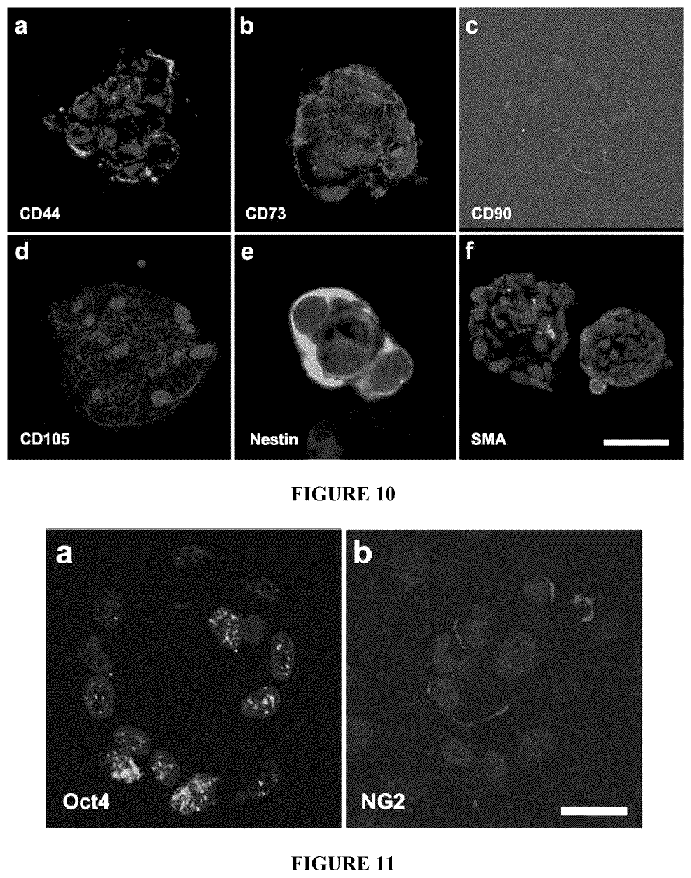

In contrast, FIG. 10 shows the positive surface marker antigen profile of human ADSCs that were cultured for four days in SFM. Immunofluorescence analysis showed that these cells were immunopositive for CD44, CD73, CD90, CD105, Nestin, and SMA. Corresponding flow cytometric analysis revealed the following relative numbers of immunopositive cells in this culture: CD44 (CD44+): 92.4%, CD73+: 99.1%, CD90+: 28.8%, CD105+: 73.3%, Nestin+: 76.2%, and SMA+: 41.2%. After 120 h in culture, the relative number of CD105+ cells increased to 96.7%. These data are in line with results reported by other authors.

Most importantly, in spheroids that were created from unmodified human ADSCs that were cultured for four days in SFM, cells expressed Oct4 as an indicator of ‘stemness’ as well as NG2 (FIG. 11) (note that no colocalization analysis was performed). This indicates the natural presence of (vascular associated) stem cells in the body without the need for prior genetic modification of the cells as in case of iPS cells (as explained in detail above the results shown in FIG. 11 cannot be explained by contamination with circulating cells that express CD34 and CD45).

It should be noted that the expression of Oct4 and NG2 in stem cells isolated from vessel walls was also reported by other authors; in the latter study, these cells were isolated from human post-mortem arterial segments that were stored in a tissue-banking facility for at least five years.

Our results further illustrate the pluripotency of vaPS cells. It has been questioned whether adult pluripotent stem cells (as defined above) exist, or if the differentiation into the three germ layers is based on the presence of a composition of different progenitor cells that are responsible for the individual differentiation capacity into the respective lineage. To answer this question, we performed two key experiments.

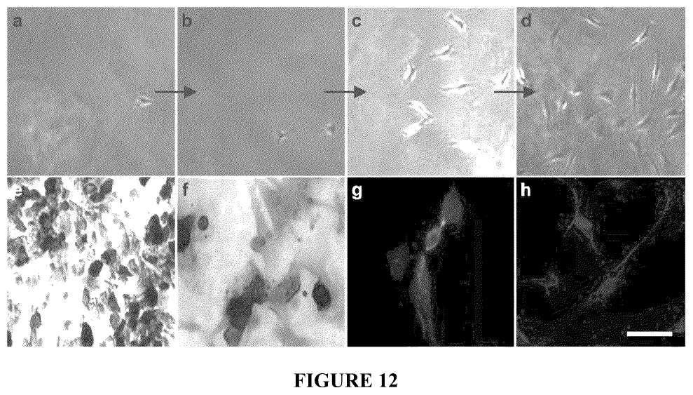

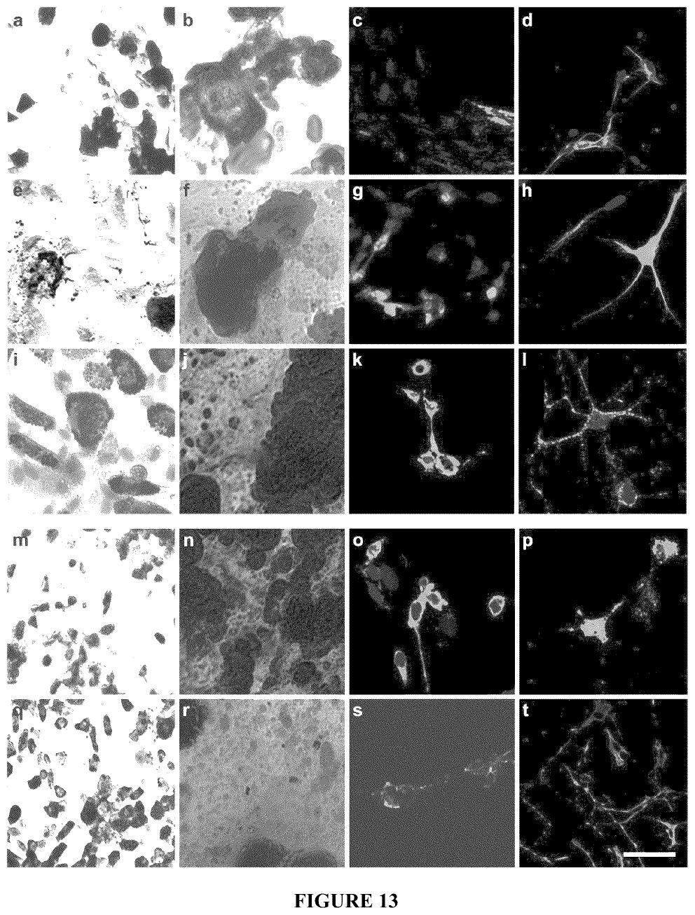

In the first key experiment, a single human vaPS cell (i.e., a single human ADSC) was clonally expanded for five days in FBS, resulting in proliferation at a doubling time of about 24 h into millions of cells (FIG. 12a-d). Then, cells were separated from this clonally expanded culture and subjected to adipogenic, osteogenic, hepatogenic, and neurogenic induction media. It was found that the clonally expanded cells, which all expressed the same genotypic profile, were able to differentiate into ectoderm, mesoderm, and endoderm (FIG. 12e-h), indicating that the initial single cell was indeed an adult stem cell with pluripotent potential.

In the second key experiment, we isolated vaPS cells from different organs (adipose tissue, heart, skin, bone marrow, and skeletal muscle) of rats and subjected them after proliferation in FBS to adipogenic, osteogenic, hepatogenic, and neurogenic induction media. Again, the cells were able to differentiate into ectoderm, mesoderm, and endoderm (FIG. 13). The results of these key experiments provide significant support for the hypothesis of a universal, vascular-associated stem cell with pluripotent potential.

### The Three Germ Layer Differentiation Potential of Human Adipose-Derived Stem Cells

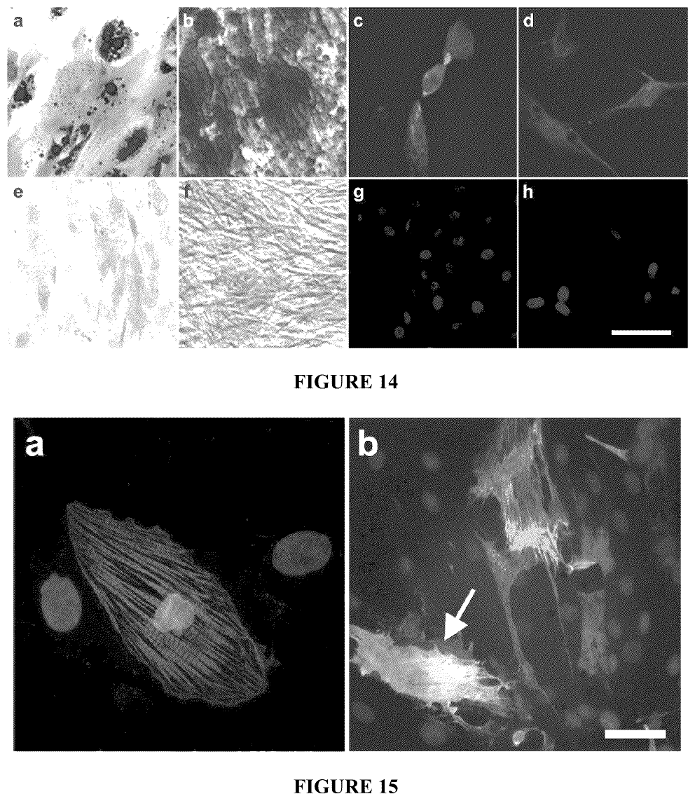

In 2007, we initially demonstrated the three germ layer differentiation potential of human ADSCs into adipocytes, osteoblasts, hepatocytes, and neurons. While the cells cultured in non-inductive media did not attain the lineage specific expression, cells subjected to the specific induction media demonstrated the respective differentiation (FIG. 14).

Integration of vaPS Cells into Host Tissue Upon Activation

The first experiments we conducted in this regard were carried out to highlight the possible influence of the microenvironment surrounding the vaPS cells. This involved co-culturing of neonatal rat cardiomyocytes with human ADSCs together with fusion-inducing hemagglutinating virus of Japan (HVJ). In order to discriminate between the two different types of cells, we labeled the human ADSCs with green fluorescent protein (GFP). FIG. 15a shows a cell that is immunopositive for the cardiac-specific protein, troponin T (red signal). FIG. 15b shows GFP (green signal) in the cytosol of human ADSCs (after treatment with HVJ and, after five days of culture time, we observed a fusion efficiency of approximately 20%). One cell shows a yellowish cell body (arrow) that was obtained by the overlay of the green signal (GFP) with a red signal originating from immunofluorescence detection of MF20, one of the early markers of the cardiomyogenic pathway differentiation. In contrast, ADSCs that were not co-cultured with rat cardiomyocytes did not show this early cardiomyogenic pathway differentiation.

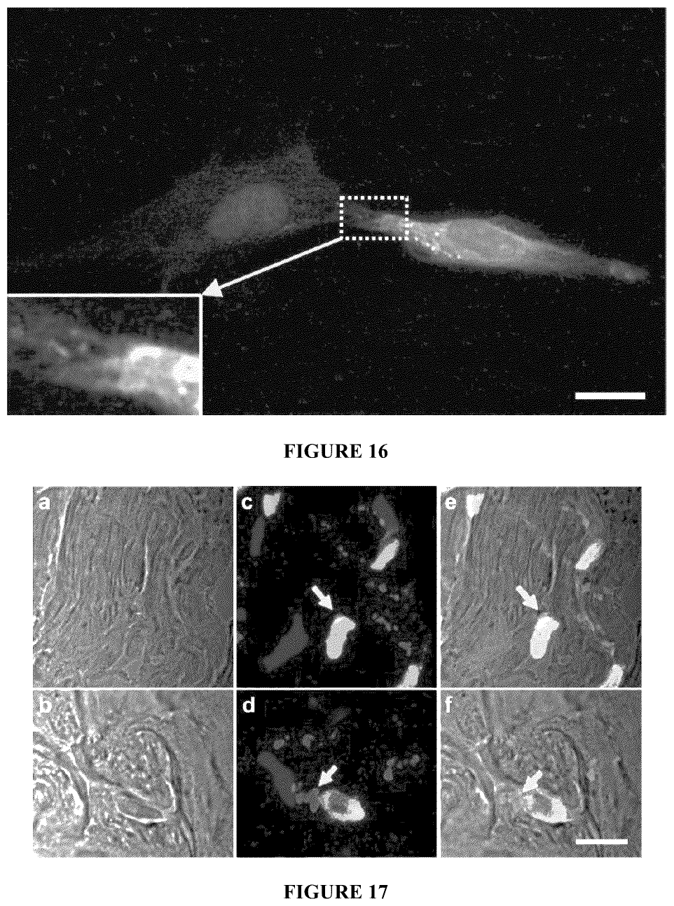

FIG. 16 shows a direct cell-cell contact between a rat ardiomyocyte (identified by immunofluorescence detection of troponin T) and a cell that expressed both GFP and troponin T. The latter represents a human ADSC at the latest stage of culturing. At higher magnification (inset in FIG. 16), it appears that the two cells adhered to each other.

We also demonstrated that five days after treatment with fusion-inducing HVJ, human ADSCs that were fused with rat cardiomyocytes showed spontaneous rhythmic contraction and exhibited action potential.

In order to demonstrate that both ADSCs and ADRCs integrate into host tissue after transplantation in vivo and form adequate contacts with cells of the host tissue, we experimentally induced myocardial infarction in severe combined immunodeficient (SCID) mice and injected human ADRCs or human ADSCs into the peri-infarct region. Four weeks later, the myocardial function was improved (evidenced by improved mean ejection fraction (p<0.01) and reduced mean end-systolic volume (p<0.01) compared to injection of saline). At that time, grafted ADRCs and ADSCs had undergone cardiomyogenic differentiation, as indicated by expression of connexin 43 and troponin I in a fusion independent manner (FIG. 17).

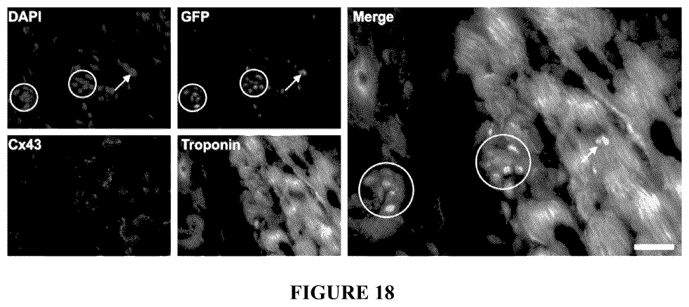

Finally, in order to demonstrate that ADSCs or their descendants differentiate into functional cells of the host tissue in vivo, we induced myocardial infarction in pigs by experimental occlusion of the left anterior descending (LAD) artery for three hours, followed by the delivery of eGFP-labeled autologous ADSCs into the balloon-blocked LAD vein (matching the initial LAD occlusion site) at four weeks after occlusion of the LAD. Six weeks later, the animals were sacrificed and sections of the heart were stained with DAPI and processed for immunofluorescence detection of GFP, connexin 43, and troponin. Cell nuclei immune positive for GFP were found in the wall of small vessels as well as in cardiomyocytes (FIG. 18).

Pigs that were treated identically except for injection of ADRCs (which cannot be labeled by definition) instead of ADSCs showed statistically significant improvements in cardiac function and structure as well, compared to the injection of saline.

Exchange of Information Between vaPS Cells and Other Cells in Cell Culture

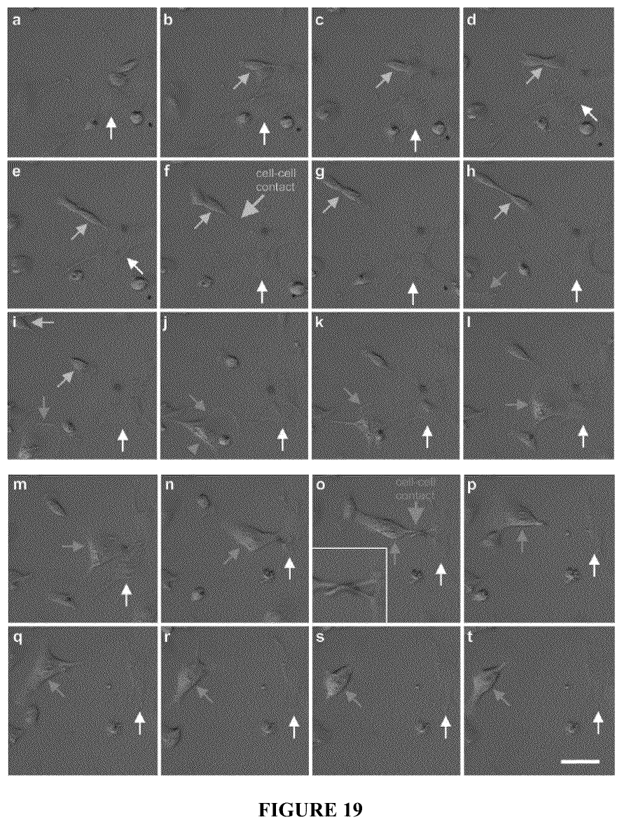

We also investigated the exchange of information between vaPS cells (i.e., ADSCs) and other cells in cell culture by time-lapse video microscopy of human ADSCs that were labeled with red quantum dots and were co-cultured with MDA-MB-231 cells (a commercially available human breast cancer cell line) that were labeled with GFP (FIG. 19). In our opinion, the communication mechanism between cells found in this experiment is the same as the cell-cell connection of endothelial progenitor cells with cardiac myocytes by nanotubes described in as an important mechanism for cell fate changes.

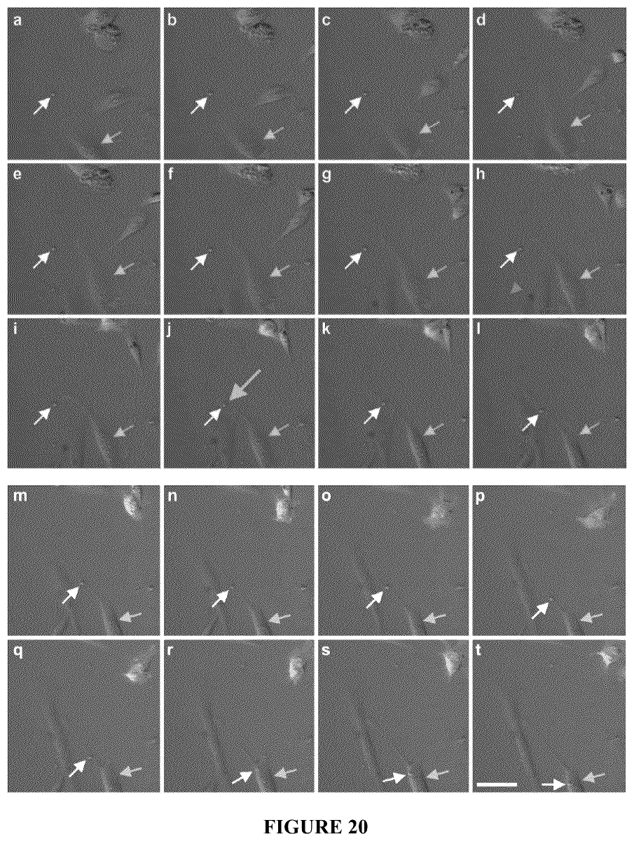

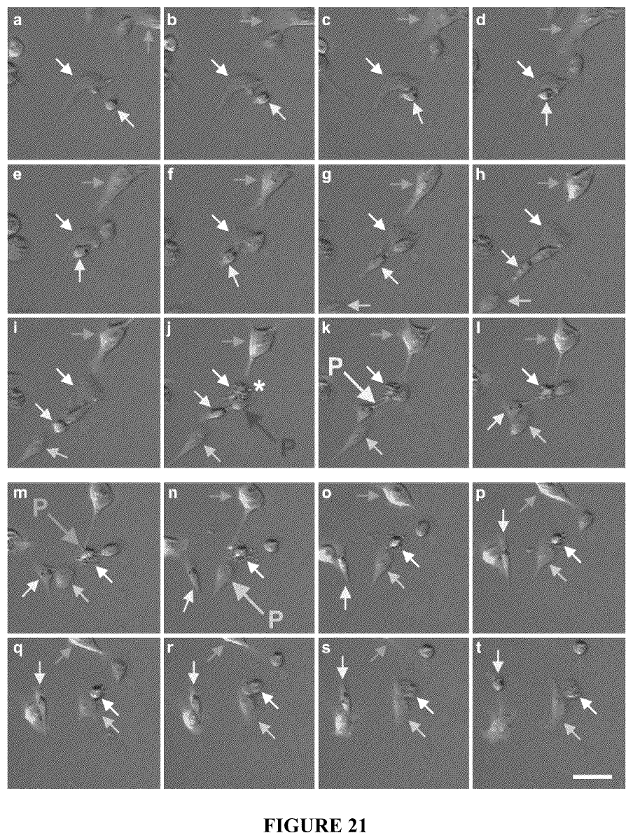

A second type of cell-cell communication is based on genetic information contained in exosomes (or microsomes), which are released from the cell surface (c.f. the inset in FIG. 2) and, upon uptake by pinocytosis (FIG. 20), induce a certain epigenetic reprogramming in the recipient cell. These exosomes are currently of major interest with respect to further elucidating cell-cell communication. Specifically, communication through the content of exosomes is considered an important factor for orientation of cells. Several micro-RNAs, transcription factors, and cytokines were identified to be involved in the transfer of epigenetic reprogramming of cells in order to direct them into a specific lineage differentiation, further supporting the hypothesis that the differentiation pathway of vaPS cells upon activation is controlled by signaling from the cells' new microenvironment.

### Immunosuppressive and Anti-Inflammatory Activities of ADSCs

ADSCs exhibit potent immunosuppressive and anti-inflammatory activities and exosomes were shown to play an important role in these processes. In recent years, apoptotic bodies, a major class of extracellular vesicles released as a product of apoptotic cell disassembly, have become recognized as another key player in immune modulation. A recent study demonstrated that apoptosis in human bone marrow-derived MSCs induced recipient-mediated immunomodulation in vivo. On the other hand, even in tissues with high cellular turnover, apoptotic cells are rarely seen because of efficient clearance mechanisms, including the sensing of cells that undergo apoptosis via ‘find me’ signals (i.e., chemotactic factors). One of these chemotactic factors is the phospholipid known as lysophosphatidylcholine. Increased concentration of lysophosphatidylcholine was found in the medium in which hematopoietic progenitor cells underwent apoptosis following growth factor withdrawal. Our time-lapse video microscopic investigations showed that human ADSCs that undergo apoptosis also stimulate the migration of other cells to the apoptotic cell, and these other cells can then take up the apoptotic bodies via pinocytosis (FIG. 21). However, it is currently unknown which ‘find me’ signals are used by ADSCs that undergo apoptosis. In any case, apoptosis of ADSCs may be a key event in immunosuppressive and anti-inflammatory activities mediated by these cells.

### A Key Role of Stem Cell Therapy in Re-Establishing Tissue Homeostasis Between Dying and Replacing Cells

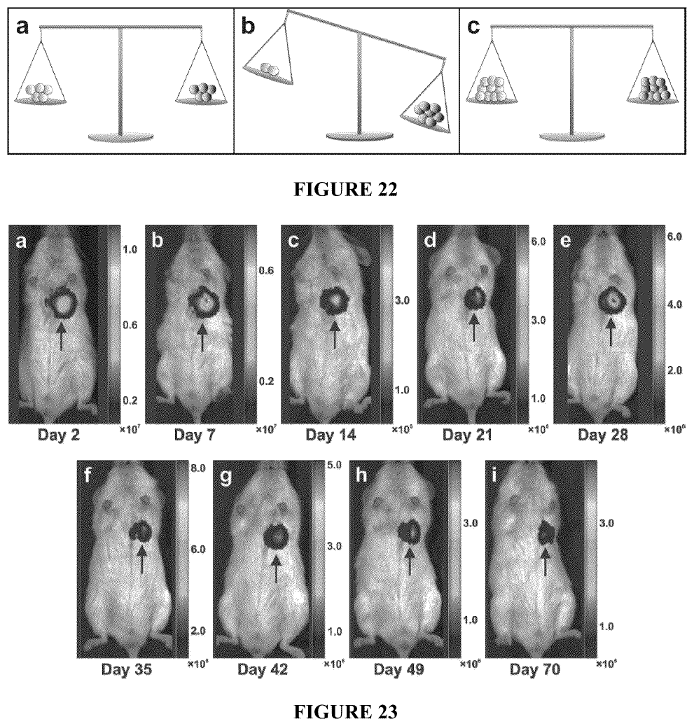

A key function of stem cells in the adult body is to contribute to the homeostasis of tissue resident parenchymal cells. As we age, there is a continuous turnover in almost every tissue between dying and replacing cells (with the exception of some nerve cells in the brain, which will not be discussed in detail here). For a long time our body can maintain tissue homeostasis; the equilibrium between dying cells and stem cells is depicted in FIG. 22a. However, tissue homeostasis can be disturbed with increasing age in all tissues, such as tendons, bone, joints, heart, liver, kidneys, and muscles, in a way that the parenchymal cells, which are responsible for the organ function, are more frequently replaced by mesenchymal fibroblastic cells. This is due to a lack of renewing power, especially if ischemia, infections, accidents, and other inflammatory or traumatic events accelerate the tissue turnover (FIG. 22b). A good example are chronic wounds that show a number of problems, including insufficient levels of cell proliferation, increased cell senescence/apoptosis, impaired angiogenesis/neovascularization, inflammation, increased production of matrix metalloproteinases (MMPs), increased matrix degradation, and decreased production of extracellular matrix.

An important question that has remained in this regard is the following: why do injured organs (for example, a heart after myocardial infraction) not source the stem cells from a part of the body where they are present and would not be ‘missed’ after recruitment? It is currently difficult to provide a satisfactory answer to this question, because it would require analyzing model organisms in which spontaneous recruitment of stem cells from other sites of the body (where they are present in larger numbers and would not be ‘missed’ after recruitment) would occur. To our knowledge, such model organisms are currently less studied. Nevertheless, it might be postulated that the release and activation of dormant stem cells from their local position within vessel walls by the osteonectin signaling (as proteins are cleaved relatively fast by proteases) might be primarily confined to the immediate vicinity of the initial stem cell location.

Several animal models for the study of limb regeneration have been described, among them the Mexican axolotl (Ambystoma mexicanum). It turned out that, in limb regeneration, a morphologically uniform intermediate (the so-called blastema) is formed, consisting of a variety of stem and progenitor cells originating from a variety of tissues. Further deciphering the genetic and molecular regeneration inducers that are involved in limb regeneration may serve as the basis to understand which signals could, in general, be used by injured organs to recover stem cells from parts of the body where they are not ‘missed’ after recruitment, followed by investigations into why this does not happen in the human body. In any case, the distance between the stem cells in these parts of the body where they would not be ‘missed’ after recruitment (as in adipose tissue) and those cells that ‘call’ for the stem cells by the release of cytokines may simply be too long.

Stem cell therapy is to be considered as the principal of transferring concentrated stem cells, which have been taken from one part of the body where they are not ‘missed’, to tissue in need of regeneration, in order to re-establish tissue homeostasis (FIG. 22c). The isolation of stem cells from suitable tissue (such as adipose tissue) and their application to other injured tissue and organs can be interpreted as the most gentle and natural approach to help the body in self-repair by increasing the number of stem cells at a location where they are exhausted, but most needed. From these considerations, it also becomes clear that stem cell therapy is not only directed to a specific organ, tissue or disease, but will take the function of replacing and repairing tissue and organs that suffer from a lack of repair, renewal, and rejuvenation.

### The Significance of Vaps Cells and Ips Cells for the Practice of Medicine

The results presented above challenge the somewhat incorrect public belief that naturally no cells would exist in the adult body that are able to differentiate into all three lineages without being first (genetically) modified. The latter may have substantially contributed to the euphoria around iPS cells (in which first an artificial (induced) overexpression of embryonic genes, such as Oct4, Klf4, Sox2, and/or cMyc is necessary), which resulted in granting Dr. Shin′ya Yamanaka (University of Kyoto, Japan) the Nobel Prize in Medicine 2012. One key motivation of this paper was to summarize evidence demonstrating that there are indeed cells in the adult body that are able to differentiate into all three lineages (i.e., the vaPS cells), and, at this time, there is no evidence that vaPS cells could not develop into all cells of the adult body. Hence, iPS cells may not be required for the general practice of medicine.

Furthermore, there are concerns that iPS cells can demonstrate features similar to cancer cells. Dr. Paul Knopfler and his team at UC Davis School of Medicine (Davis, CA, USA) were the first to demonstrate that induced pluripotency and oncogenic transformation are related processes when comparing the transcriptomes of iPS cells with the transcriptomes of cancer cells. Hence, the iPS cells technology will most likely not advance to a stage where therapeutic transplants are necessarily deemed safe. On the other hand, iPS cells are supportive in helping to better understand differentiation pathways of stem cells and patient-specific bases of diseases, as well as to develop personalized drug discovery efforts.

In light of these considerations, the clinical significance of vaPS cells is outlined in detail in the following sections.

### Key Advantages of Adipose-Derived Regenerative Cells and Adipose-Derived Stem Cells for Cell Based Therapies

Comparison of Bone Marrow Derived Stem Cells with Adipose-Derived Regenerative Cells and Adipose-Derived Stem Cells

For almost a decade bone marrow was the primary source of stem cells for research into and development of therapies based on stem cells isolated from the adult body. Bone marrow-derived stem cells exhibit significant potential for promoting tissue regeneration, protection of ischemic tissue at risk, and modulation of inflammation and autoimmunity. However, utilizing bone marrow-derived stem cells for therapeutic purposes typically requires to first isolate these cells and expand them in culture. Because of the overwhelming presence of hematopoietic progenitor cells in bone marrow that aim to form new blood cells, only a small fraction of the cells in fresh bone marrow aspirate are stem cells. In contrast, other tissues, such as adipose tissue, yield orders of magnitude higher numbers of stem cells per unit volume than bone marrow. Thus, ADRCs may be utilized as a fresh cell preparation, rich in vaPS cells, without the need for expansion in cell culture.

Compared to other sources of stem cells in the adult body, adipose tissue has the following specific advantages: (i) adipose tissue is readily available in most individuals; (ii) small amounts of adipose tissue (25 to 100 mL) can be harvested using a mini-liposuction procedure with low invasiveness, with tolerable discomfort and low donor-site damage; (iii) considerably larger amounts of stem cells can be obtained from adipose tissue than from the same amount of bone marrow; and (iv) ADRCs can be used in clinical applications without further need of culturing (as in case of ADSCs). Given these advantages, unmodified, autologous ADRCs (UA-ADRCs) appear to be the most promising candidate for repair and regeneration of many tissues, including chronic wounds, soft tissue defects, bone and cartilage defects, non-healing fractures, injured tendons, diseased or injured myocardium, urological conditions such as incontinence, and neurological conditions.

Difference in the Effectiveness of Various Systems and Methods that are Available for Isolating Adipose-Derived Regenerative Cells

Different techniques and protocols were described for releasing ADRCs for therapeutic use. Collagenase I and II containing enzyme preparations that degrade collagen are commonly used. However, in order to release the vaPS cells from their binding site in the extracellular matrix inside the blood vessels (and hereby to release the cells from their ‘hibernating’ or silenced state), collagenases are only partially effective. The addition of a neutral protease to a collagenase enzyme preparation can significantly increase the number of ADRCs recovered from a given volume of adipose tissue. This was achieved by developing the proprietary Matrase® enzymatic reagent (InGeneron Inc., Houston, TX, USA). Isolating ADRCs with the Matrase enzymatic reagent and the Transpose RT® system (InGeneron) appears advantageous to other commercial cell separation systems. Specifically, ADRCs that were isolated with the Matrase enzymatic reagent and the Transpose RT system may contain approximately 40% of cells that are immunopositive for CD29 and CD44, which are markers of ADSCs. The latter authors also reported a colony-forming units frequency (CFU-F) (considered to be an indicator of stemness) of approximately 11% of ADRCs isolated with the Matrase enzymatic reagent and the Transpose RT system. Other authors reported relative CFU-F values of approximately 8% when isolating ADRCs from equine adipose tissue using the same technology. In contrast, relative CFU-F values between 0.2% and 1.7% were reported for ADRCs that were isolated in head-to-head comparisons with four other commercial cell separation systems that do not make use of neutral protease. However, a direct comparison of the CFU-F values is hardly possible due to significant methodological differences surrounding how the respective CFU-F values were determined.

Long-Term Survival of Adipose-Derived Stem Cells after Transplantation in Animal Models into the Heart and Subcutaneous Locations

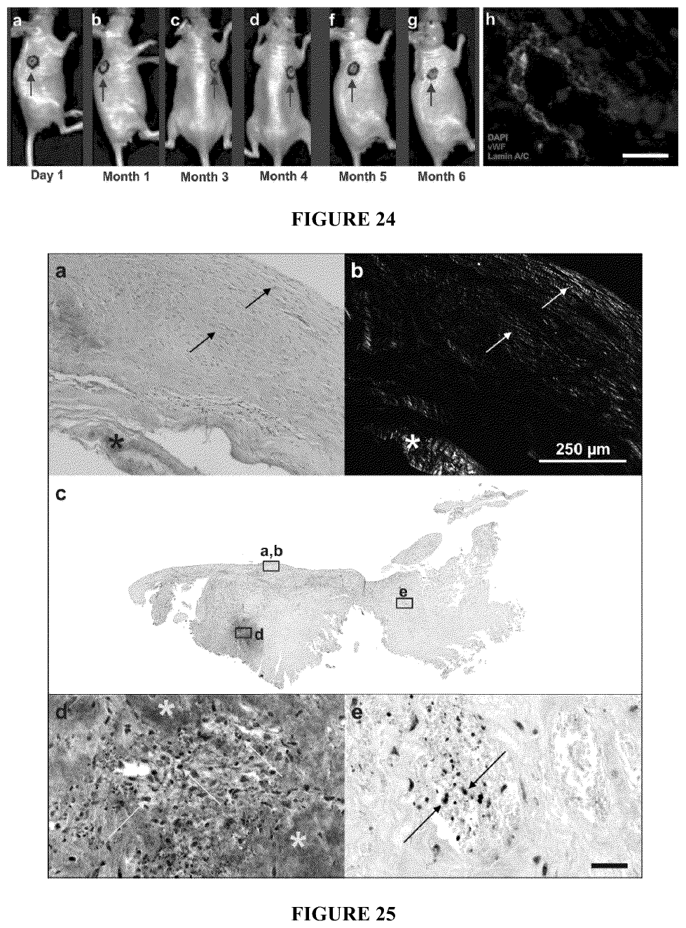

In order to study the capacity of ADSCs to survive for a long time at the site of engraftment, we transfected human ADSCs with a lentiviral vector expressing GFP and luciferase (when the luciferase enzyme is expressed in living cells, these cells are capable of converting systemically injected luciferin dye into an active luminescent fluorophor that can be detected noninvasively). FIGS. 23 and 24 show the time course of luciferin positive human ADSCs that were either intramyocardially delivered into SCID mice after experimental induction of myocardial infarction by permanent ligation of the left anterior descending coronary artery (FIG. 23), or into a subcutaneous location of SCID mice (FIG. 24), respectively. Strong bioluminescence signals were found over the injection sites at all investigated time points (up to 70 days post-delivery in FIG. 23 and up to six months post-delivery in FIG. 24; further time points were not investigated), demonstrating the long-term survival of ADSCs delivered into injured hearts or at subcutaneous locations, respectively. At no other location of the body of the SCID mice, a cell engraftment was detected.

To better understand the fate of the delivered ADSCs, we investigated subcutaneous tissue harvested at the injection site at four weeks after subcutaneous application of human ADSCs. Immunofluorescence detection of von Willebrand factor (red signal in FIG. 24h) and Lamin A/C (green signal in FIG. 24h) demonstrated that the human ADSCs delivered into a subcutaneous location of a SCID mouse participated in the formation of new blood vessels.

In the heart of the mice shown in FIG. 23, we made the following observations 70 days post-delivery (all corresponding images are provided in Bai et al.: some injected human ADSCs had differentiated into cardiomyocytes, as evidenced by co-expression of troponin I and lamin A/C (detecting human cell nuclei) in those cells (c.f. FIG. 17); other injected human ADSCs had differentiated into endothelial cells, as evidenced by coexpression of endothelial cell marker vWF and lamin A/C; some injected human ADSCs maintained proliferation potential, as evidenced by co-expression of proliferating cell marker Ki67 and lamin A/C; and some injected human ADSCs underwent apoptosis, as evidenced by the presence of TUNEL-positive signals in lamin A/C positive human ADSCs.

Moreover, no lamin A/C signal was observed in sections of the lung, liver, kidney, spleen, and brain of these mice, indicating that intramyocardially delivered human ADSCs did, in principle, not migrate into other tissues or organs.

### Specific Therapeutic Benefits of Adipose-Derived Regenerative Cells

One of the most striking features of ADRCs in cell-based therapy is their differentiation potential without any prior manipulation, genetic alteration, or the need for culturing the cells. The latter facilitates to isolate ADRCs and re-apply them to the same subject at the point of care without the need for expensive equipment, complicated processing, or repeated interventions.

It is crucial to bear in mind that, in contrast to ADSCs, UA-ADRCs in principle cannot be labeled (because this would render them modified). Accordingly, it is not possible to experimentally determine whether the following benefits of ADSCs also apply to UA-ADRCs (although it is reasonable to hypothesize that this is indeed the case). Specifically, ADSCs can (i) stay locally, survive, and engraft in the new host tissue into which the cells were applied (FIGS. 17 and 18); (ii) differentiate under guidance of the new microenvironment into cells of all three germ layers (FIGS. 12-14); (iii) integrate into and communicate within the new host tissue by forming direct cell-cell contacts (FIGS. 16-18); (iv) exchange genetic and epigenetic information through release of exosomes (FIG. 19); (v) participate in building new vascular structures in the host tissue (FIG. 24h and D'Ippolito et al.); (vi) positively influence the new host tissue by release of cytokines (among them vascular endothelial growth factor and insulin-like growth factor 1); (vii) protect cells at risk in the new host tissue from undergoing apoptosis; and (viii) induce immune modulatory and anti-inflammatory properties, whereby the inhibiting effect on apoptosis may play an important role.

Local vs. Systemic Application of UA-ADRCs

Several studies showed that local injection of ADRCs is safe.

When stem cells are injected into the circulation, they are ‘searching’ for a place where they could be of benefit. As a tumor is considered a ‘wound that does not heal’, it releases cytokines and other factors that aim to attract circulating stem cells to the tumor site, where stem cells may assist the tumor to build its stroma and thereby help the tumor to grow faster. Hence, UA-ADRCs preferably should be applied locally to the side of need. In case of systemic application, the potential of UA-ADRCs to support an already existing tumor in its growth (in contrast to the absent ability of UA-ADRCs to induce a de novo tumor) should be considered, pointing to the need for evaluation of the oncogenic status of the patient prior to a systemic application.

### EXAMPLES OF APPLICATION OF UA-ADRCS IN REGENERATIVE CELL THERAPY

### Example 1: Tendon Defects

Our group recently published a prospective, randomized, controlled first-in-human pilot study suggesting that the use of UA-ADRCs in subjects with symptomatic, partial-thickness rotator cuff tear (sPTRCT) is safe and leads to improved shoulder function without adverse effects [127]. Specifically, we demonstrated that the risks connected with treatment of sPTRCT with UA-ADRCs were not greater than those connected with treatment of sPTRCT with corticosteroid injection. On the other hand, the subjects who were treated with UA-ADRCs showed a statistically and significantly higher mean American Shoulder and Elbow Surgeons Standardized Shoulder Assessment Form (ASES) total scores at 24 weeks and 52 weeks post-treatment than those subjects who were treated with corticosteroid. Based on the encouraging results of this pilot study, a respective pivotal, randomized controlled trial on 246 patients suffering from sPTRCT is currently ongoing.

Furthermore, we investigated a biopsy of a human supraspinatus tendon that was taken ten weeks post-treatment of a traumatic sPTRCT using UA-ADRCs. Most intriguingly, the microscopic images of the tendon treated with UA-ADRCs clearly demonstrated that a different type of healing had taken place. Specifically, the formation of new tendon tissue and the absence of scar tissue (FIG. 25a,b) that was observed are regenerative processes that are typically only observed in fetal tendons. We also found abundant intracellular and extracellular presence of tenomodulin in a region that most probably represented the site of injection of UA-ADRCs (FIG. 25c,d) (tenomodulin is a tendon-specific marker important for tendon maturation, with key implications for residing tendon stem/progenitor cells and the regulation of endothelial cell migration). This finding was in line with the hypothesis that tendon regeneration observed in the investigated biopsy was ‘orchestrated’ from this region. Furthermore, the presence of tenomoduline immunopositive cells inside microvessels in another region of the biopsy (that mostly showed degenerative tendon tissue, characterized by unorganized collagen and few, rounded cells) (FIG. 25e) may have indicated an unsuccessful attempt of the body to endogenously initiate tendon regeneration by transferring corresponding cells via the blood stream into the injured/degenerative tissue.

Collectively, these data strongly support the usefulness of transferring UA-ADRCs, which have been taken from one part of the body where they are not ‘missed’ to tissue in need of regeneration, in order to re-establish tissue homeostasis (c.f. FIG. 22c).

### Example 2: Cartilage Defects

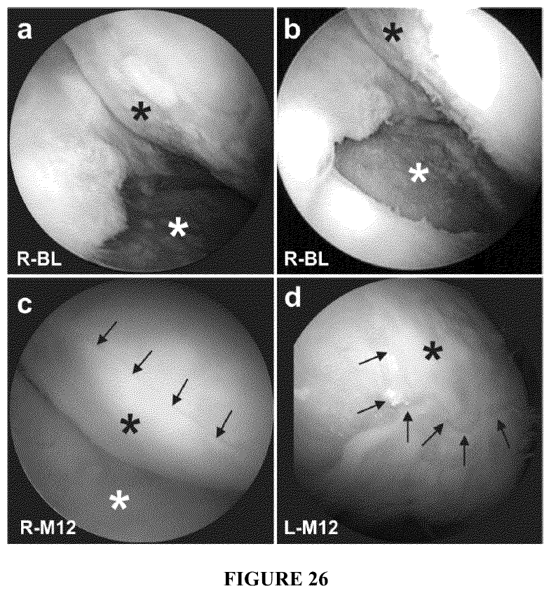

The advantages of treating cartilage defects with ADRCs were documented in 27 clinical trials so far, with a total number of >700 subjects treated with ADRCs (Schmitz et al.; systematic review and meta-analysis in preparation). These specific advantages are exemplified here by the following example of a male, 51-year-old subject who presented with recurring and increasing pain in both knee joints during walking and other activities (all treatments and procedures described in this section were performed in the framework of a clinical study that was approved by the Freiburg Ethics Commission International (feki; Freiburg, Germany) (feki code 013/1371)). The subject's history included a tibial chondrocyte transplant that had been performed three years previously. FIG. 26a shows an arthroscopic view of third-degree damage to the right tibial plateau where the transplanted chondrocytes were gone and only the artificial matrix with small holes implanted on the tibial plateau was still present (white asterisk in FIG. 26a). Furthermore, considerable osteoarthritic damage of the femoral cartilage was observed (black asterisk in FIG. 26a). FIG. 26b shows the situation after arthroscopic removal of the failed chondrocyte transplant (white asterisk in FIG. 26b) as well as ‘mushy’ and damaged cartilage structure on the femoral condyles before it was removed (black asterisk in FIG. 26b). Then, the right knee was treated with a single application of UA-ADRCs obtained from 100 mL of lipoaspirate.

A control arthroscopy one year later showed complete healing of the tibial defect (white asterisk in FIG. 26c) and of the femoral parts, with formation of new whitish cartilage that showed a sharp demarcation border to the original, more yellowish cartilage (arrows in FIG. 26c).

The left knee of the same subject was treated with a standard procedure without application of UA-ADRCs, i.e., arthroscopic removal of damaged cartilage and drilling of small holes into the bone. A control arthroscopy one year later showed a somewhat uneven, overshooting fibroblastic scar formation (asterisk in FIG. 26d) without a sharp demarcation border to the original cartilage (arrows in FIG. 26d). This indicated that there was some sort of healing, but not a regrowth of organized cartilage, as we have hypothesized for the right knee after application of UA-ADRCs.

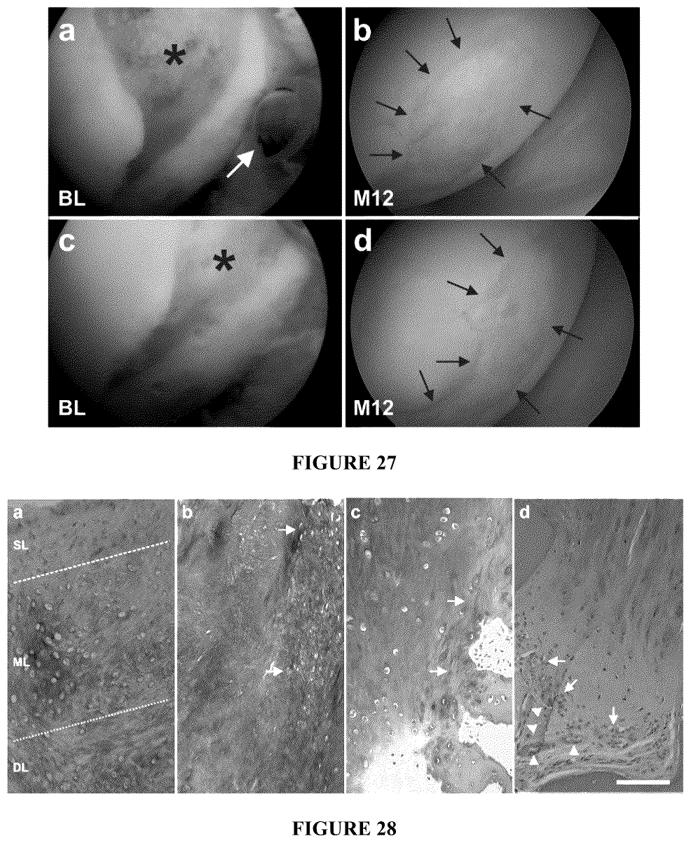

FIG. 27 shows arthroscopic views of cartilage defects of the knees of two other subjects that were also successfully treated with UA-ADRCs. Of note, the finding of a sharp demarcation border between the newly formed cartilage and the original cartilage (arrows in FIG. 26c) was found as well in these subjects one year after treatment with UA-ADRCs (arrows in FIG. 27b,d). Again, we have hypothesized that this arthroscopic finding indicated regrowth of organized cartilage.

To test the hypothesis that the sharp demarcation borders between the newly formed and the original cartilage (arrows in FIGS. 26c and 27b,d) indicated regrowth of organized cartilage after application of UA-ADRCs, we obtained written, informed consent by the subject represented in FIG. 26 which stated that small tissue samples could be taken from the regenerated tissue during the follow-up arthroscopy. Histologic analysis of the tissue samples showed two key differences between the samples. (i) After application of UA-ADRCs, the newly formed cartilage showed (such as in a textbook of histology) a zonal organization with differently shaped chondrocytes in a superficial, middle, and deep layer (FIG. 28). In contrast, without application of UA-ADRCs, a more amorphous fibrocartilage with scattered cells (arrows in FIG. 28b) was achieved that had no such layered organization (FIG. 28b). (ii) Furthermore, after application of UA-ADRCs, the contact zone between the newly formed cartilage and bone showed (again, such as in a textbook of histology) typical chondrocytes with a small nucleus and a hollow space around (arrows in FIG. 28c). In contrast, without application of UA-ADRCs, the contact zone between the newly formed cartilage and bone showed an infiltration with inflammatory cells, fibroblasts (arrows in FIG. 28d) and small blood vessels (arrowheads in FIG. 28d).

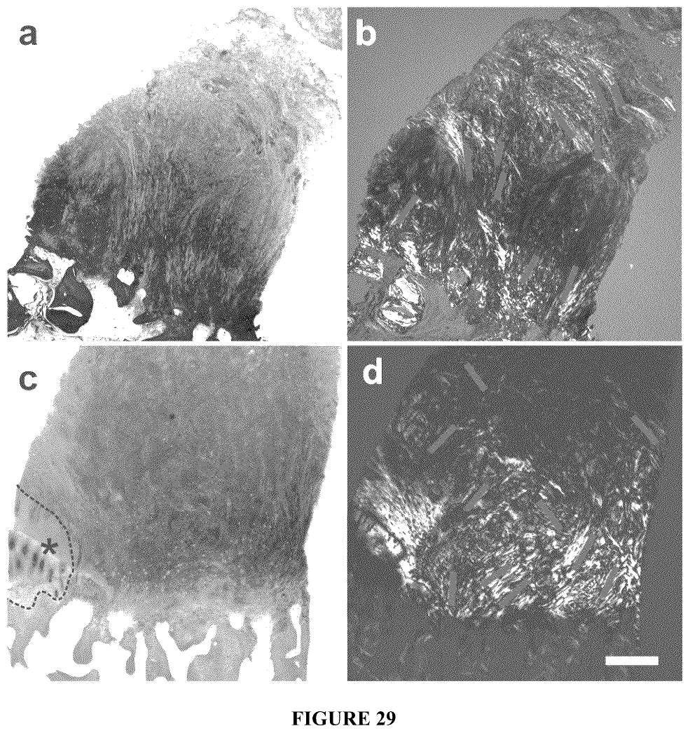

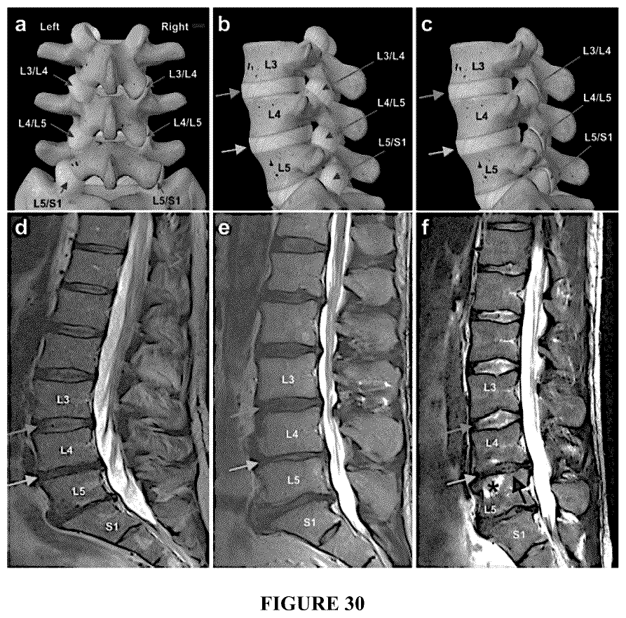

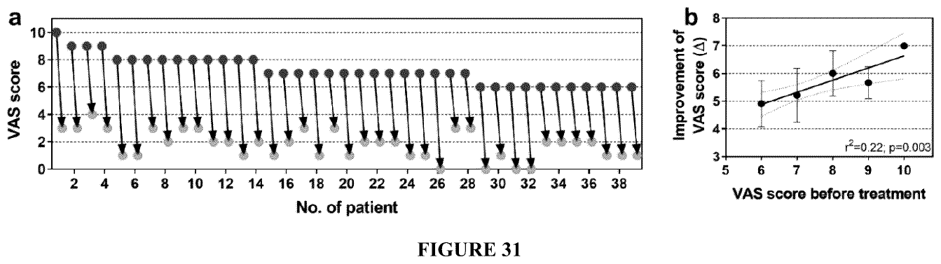

Analysis of the tissue sample taken during arthroscopic inspection of the right knee of the subject represented in FIG. 26 at one year after arthroscopic removal of damaged cartilage and a single application of UA-ADRCs with polarized light microscopy demonstrated that the collagen fiber bundles in the deep and middle layers of the newly formed cartilage showed a more vertical orientation (perpendicular to the border between bone and cartilage), whereas the collagen fiber bundles in the superficial layer showed a more horizontal orientation (parallel to the surface) (FIG. 29). This finding is in line with the description of the physiologic orientation of collagen fiber bundles in articular cartilage in the literature when analyzed with polarized light microscopy.

To our knowledge, the results presented in this section go beyond the state-of-the-art in the field of regenerating damaged cartilage with ADRCs and ADSCs. In a recent review, a number of clinical studies were listed in which cartilage defects in the human knee were treated with ADSCs. Of note, in all of these studies, cultured ADSCs were applied, whereas we have been using fresh, uncultured ADRCs (for the differences and advantages of ADRCs over ADSCs see Section 5.4). The maximum follow-up period in was only six months after application of ADSCs (MRI, arthroscopy, and histologic analysis in both studies; n=18 subjects in both studies). In the single case report, MRI was performed at twelve months after application of ADSCs, but no arthroscopy and, thus, no histologic analysis were performed. Furthermore, in none of these studies tissue samples were investigated using polarized light microscopy.

### Example 3: Chronic, Recalcitrant Low Back Pain Caused by Lumbosacral Facet Syndrome

Lumbosacral facet syndrome is a term used to describe a painful condition caused by inflammation and irritation of the zygapophyseal (facet) joints of the spine (FIG. 30a-c). It is most commonly caused by degenerative changes in the lumbosacral spine, and predominantly the consequence of a reduction in height of a lumbar disc, following a previous disc prolapse. As a consequence, the distribution of body weight (that normally rests on the vertebral column and the discs) is now also resting on the facet joints that are made for elasticity but not for weight load bearing. As a consequence of this overload, the small facet joints become chronically inflamed. Symptoms include low back pain with or without referral to the lower extremities. To our knowledge, reports on cell-based therapies for chronic low back pain caused by lumbosacral facet syndrome have not yet been published.

FIG. 30d-f shows typical wear and tear that occurred in the spine of three former professional ski racers (all treatments and procedures described in this section were performed in the framework of clinical assessment). The green arrows indicate normal intervertebral disc structure between the lumbar vertebrae L3 and L4, demonstrating that there was a certain level of water content, reflected by the white signal in the MRI. In contrast, the yellow arrows show that between vertebrae L4 and L5 in all three athletes there was a reduction in height of the intervertebral disc due to the diminished overall volume of the intervertebral disc. The increased body weight now resting on the facet joints resulted in an inflammatory reaction of the facet joints. All three athletes were treated with UA-ADRCs. The cells were injected at the site of the right and left facet joints between LA and L5 as well as between L5 and SI within a single procedure that lasted about two hours from harvesting the adipose tissue to injecting the cells. This treatment resulted in significant and long-lasting (now more than three years) pain reduction that enabled one of these athletes to very successfully return to competitive sports.

FIG. 31a shows individual pain scores on a VAS scale (with 0 representing no pain and 10 representing maximum, unbearable pain) of n=39 subjects with chronic, recalcitrant low back pain caused by lumbosacral facet syndrome before (red dots in FIG. 31a) and one year after (green dots in FIG. 31a) treatment with UA-ADRCs isolated from 100 mL of lipoaspirate each (the follow-up interval ranged between one and more than three years) (modified from (all treatments and procedures described in this section were performed in the framework of a clinical study that was approved by the Freiburg Ethics Commission International (feki; Freiburg, Germany) (feki code 016/1252)). The mean and standard error of the mean of the VAS scores before (red dots) and after (green dots) treatment was 7.21±0.17 and 1.80±0.17; this difference was highly statistically significant (Wilcoxon matched-pairs signed rank test; p<0.001). As a consequence, the quality of life of these subjects was substantially improved. There was no single subject that was not impressively benefitting from the treatment.

FIG. 31b shows the mean and standard deviation of the individual improvement of the VAS score after treatment as a function of the VAS score before treatment of these 39 subjects. Linear regression analysis showed a statistically significant relationship between the VAS score before treatment and the individual improvement of the VAS score after treatment, with the best results obtained with the highest VAS scores before treatment (r2=0.22; p=0.003). This impressively demonstrates the potential of UA-ADRCs for the treatment of chronic, recalcitrant low back pain caused by lumbosacral facet syndrome, and perhaps opens up the possibility for obtaining comparable results for other chronic pain conditions of the musculoskeletal system. A corresponding randomized controlled trial is currently ongoing [145].

### Example 4: Guided Bone Regeneration

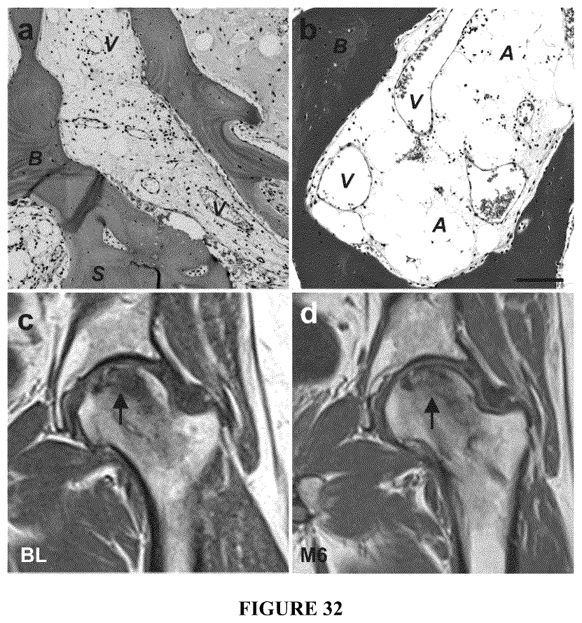

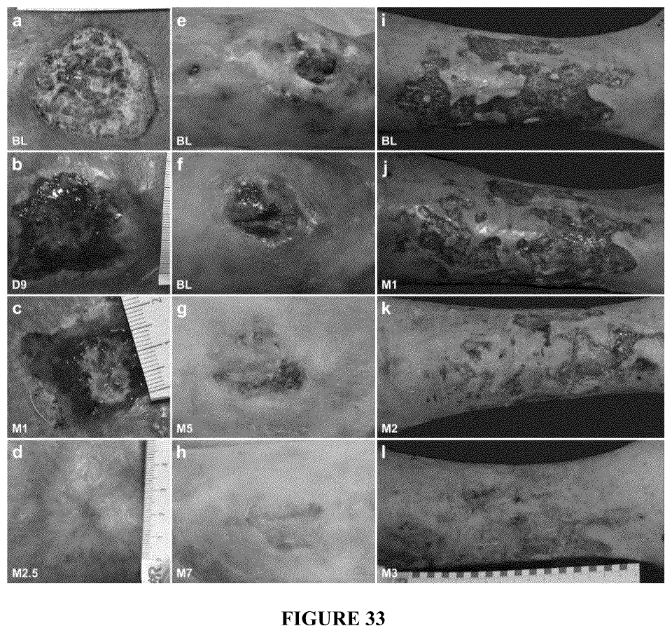

The promising results achieved in the treatment of cartilage defects and chronic, recalcitrant low back pain caused by lumbosacral facet syndrome (described in detail above) gave reason to hypothesize that the application of UA-ADRCs could also advance the treatment of other pathologies of the musculoskeletal system. In this regard, it was a key finding that, in guided bone regeneration (GBR) (exemplified by a case of a 79-year-old subject who presented with a partly failing maxillary dentition and who was treated with a bilateral external sinus lift procedure as well as a bilateral lateral alveolar ridge augmentation), the combined application of UA-ADRCs, Fraction 2 of plasma rich in growth factors (PRGF-2), and an osteoinductive scaffold (OIS) (Treatment A), was superior to the combination of PRGF-2 and the same OIS alone (Treatment B). Specifically, Treatment A resulted in faster buildup of higher relative amounts (area/area) of newly formed bone, connective tissue and arteries as well as in lower relative amounts of adipocytes and veins at 34 weeks after GBR (FIG. 32a,b) compared to standard treatment without stem cells.

Avascular necrosis of the femoral head is one of the many indications where bone regeneration is essential for rehabilitation. Cell-based therapy for this pathology has been addressed in a number of clinical studies. One of these studies was a case report of a 43-year-old male subject who was successfully treated with ADRCs mixed with platelet-rich plasma and hyaluronic acid. We went one step further and treated a 41-year-old male subject suffering from avascular necrosis of the femoral head only with ADRCs (all treatments and procedures were performed in the framework of clinical assessment). FIG. 32c shows an MRI scan of the left hip of this subject who was confined to a wheelchair because of unbearable pain during walking, with a significant necrotic space in the head of the subject's left femur. Six months after two applications of UA-ADRCs (that were locally injected through a channel which came through the lateral side of the greater trochanter), a control MRI scan showed a markedly improved situation (FIG. 32d), and the subject could leave the wheelchair and walks now without pain.

Other Treatment with UA-ADRCs

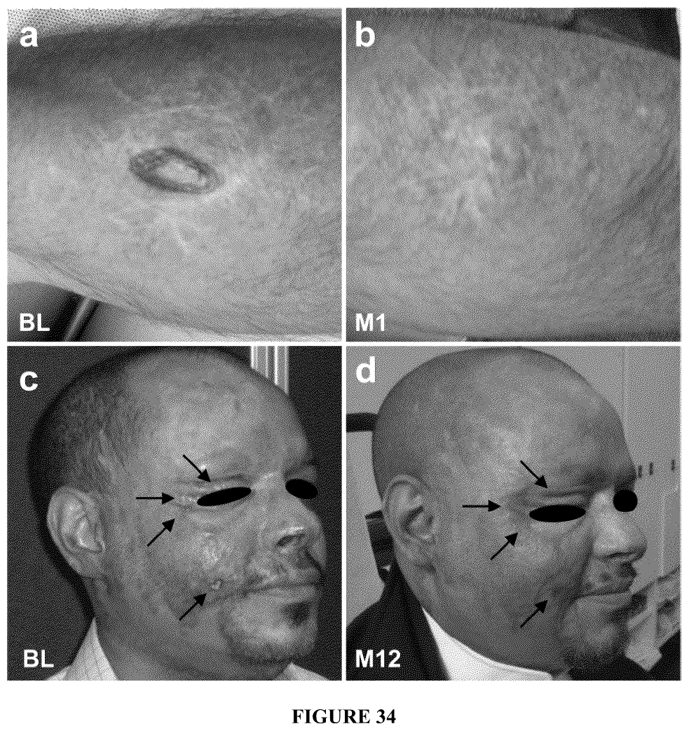

Several studies on animal models and clinical pilot studies have shown that human ADRCs and ADSCs are able to enhance and accelerate wound healing, especially in chronic wounds. An example of successful application of UA-ADRCs for treating chronic wounds in humans is shown in FIG. 33, which represent cases of prolonged wounds that did not heal for several years (these treatments and procedures were performed in the framework of a clinical study that was approved by the Ethics Committee of the Medical Faculty of the Technical University Munich (Munich, Germany) (no. 5639/12)). The underlying principle in those wounds is that the regenerative power of the local stem cells is exhausted due to the repeated futile attempts at healing. Debridement of the wounds showed that debrided tissue contained only very few living cells. The transfer of UA-ADRCs activated the wounds after a few days and induced shrinkage and significant reduction in the inflammatory redness. Typically, after two to three months, all wounds based on venous insufficiency were closed. Of note, the images shown in FIG. 33 impressively demonstrate that UA-ADRCs do not only work in younger people. In one 85-year-old subject who suffered from open leg wounds of more than 100 cm² for several years, healing was obtained after a single application of UA-ADRCs (FIG. 33c).

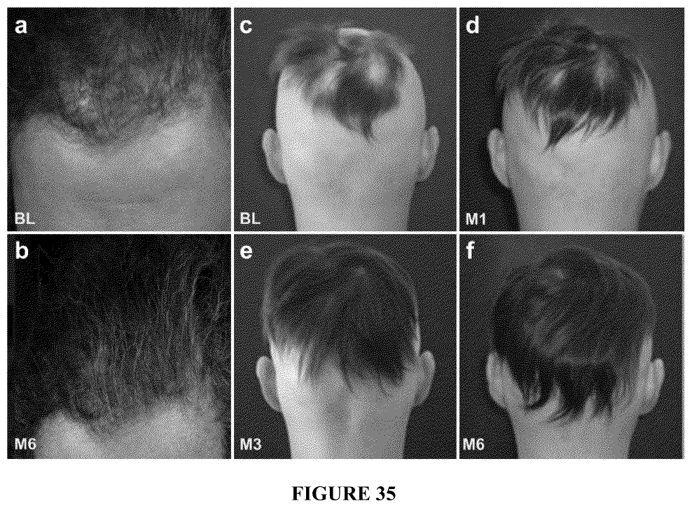

Primarily not considered a mainstream indication for stem cell therapy, there is anecdotal evidence indicating that UA-ADRCs have a great effect on remodeling of scar tissues. As demonstrated in FIG. 34, the degradation of the extracellular matrix, which leads to scarring, can be reversed (all treatments and procedures described in this section were performed in the framework of clinical assessment). We still do not fully understand the exact mechanisms behind this effect. However, the expression of MMP2 may enable ADRCs to migrate through tissue, and subsequent expression of MMP9 might be responsible for the realignment of the extracellular matrix.

FIG. 35 shows the effects of UA-ADRCs on hair growth (all treatments and procedures described in this section were performed in the framework of clinical assessment). The mechanism is assumed to be similar to the regeneration of an organ or other tissues. Specifically, we hypothesize that the delivered stem cells most likely ‘were guided’ by the remaining hair roots to their ‘local task’ of trans-differentiation into hair.

As shown in this disclosure, regenerative medicine and cell therapy are not yet part of mainstream clinical practice. Therapies based on UA-ADRCs discussed in this paper appear to be highly promising candidates for repair and regeneration of many tissues and ultimately for wide adoption to the practice of medicine. One of the most striking features of UA-ADRCs is their differentiation potential without any prior modification or need for culturing. Furthermore, UA-ADRCs can be obtained from a small amount of adipose tissue when using the appropriate, enzyme-supported technology for isolating vaPS cells. The fact that tissue can be harvested from and cells can be re-applied to the same subject at the point of care in one clinical session without the need for expensive equipment, complicated processing, or repeated interventions indicates easy integration into the clinical workflow.

As with any medical innovation, the scientific and medical community interested in these novel therapies needs to develop sound clinical evidence to further show safety and efficacy of cell-based therapies. Our understanding of the mechanism of actions and potential benefit of stem cell therapy has increased enormously over the past decade and we hope that there is now enough data to convince others to embark on scientifically designed clinical studies that will provide the necessary objective evidence. Especially musculoskeletal indications with their large incidence and prevalence rates and often substantial total cost of care associated with current clinical practice should prove to be attractive candidates for such efforts.

An important factor for successful implementation of therapies using UA-ADRCs will be the proactive support of regulatory authorities to design frameworks that, while addressing valid concerns around the safety of unproven therapies that can be found in some places currently, show a clear path to market approval and reimbursement.

As shown above, this disclosure provides a novel discovery of vascular-associated pluripotent stem cells (vaPS) that can be readily obtained from different parts of an human body, and can be applied in stem cell therapies without prior manipulation or modification or culturing.

The following references are incorporated by reference in their entirety for all purposes.
1. Friedenstein, A. J.; Petrakova, K. V.; Kurolesova, A. I.; Frolova, G. P. Heterotopic of bone marrow. Analysis of precursor cells for osteogenic and hematopoietic tissues. Transplantation 1968, 6, 230-247.
2. Friedenstein, A. J.; Deriglasova, U. F.; Kulagina, N. N.; Panasuk, A. F.; Rudakowa, S. F.; Luriá, E. A.; Ruadkow, I. A. Precursors for fibroblasts in different populations of hematopoietic cells as detected by the in vitro colony assay method. Exp. Hematol. 1974, 2, 83-92.
3. Nature Portfolio Stem Cells. Available online: https://www.nature.com/subjects/stem-cells (accessed on 6 Aug. 2021).
4. Stem Cell Key Terms. Available online: https://www.cirm.ca.gov/patients/stem-cell-key-terms #2 (accessed on 6 Aug. 2021).
5. Types of Stem Cells. Available online: https://www.closerlookatstemcells.org/learn-about-stem-cells/types-of-stem-cells/#mesenchymal (accessed on 6 Aug. 2021).
6. Boland, M. J.; Hazen, J. L.; Nazor, K. L.; Rodriguez, A. R.; Gifford, W.; Martin, G.; Kupriyanov, S.; Baldwin, K. K. Adult mice generated from induced pluripotent stem cells. Nature 2009, 461, 91-94.
7. Zhao, X.; Li, W.; Lv, Z.; Liu, L.; Tong, M.; Hai, T.; Hao, J.; Guo, C.; Ma, Q.; Wang, L.; et al. IPS cells produce viable mice through tetraploid complementation. Nature 2009, 461, 86-90.
8. Dominici, M.; Le Blanc, K.; Mueller, I.; Slaper-Cortenbach, I.; Marini, F. C.; Krause, D. S.; Deans, R. J.; Keating, A.; Prockop, D. J.; Horwitz, E. M. Minimal criteria for defining multipotent mesenchymal stromal cells. The International Society for Cellular Therapy position statement. Cytotherapy 2006, 8, 315-317.
9. Bai, X.; Yan, Y.; Song, Y.-H.; Seidensticker, M.; Rabinovich, B.; Metzele, R.; Bankson, J. A.; Vykoukal, D.; Alt, E. Both cultured and freshly isolated adipose tissue-derived stem cells enhance cardiac function after acute myocardial infarction. Eur. Heart J. 2010, 31, 489-501.
10. Winnier, G. E.; Valenzuela, N.; Peters-Hall, J.; Kellner, J.; Alt, C.; Alt, E. U. Isolation of adipose tissue derived regenerative cells from human subcutaneous tissue with or without the use of an enzymatic reagent. PLOS ONE 2019, 14, e0221457.
11. Davidoff, M. S. The pluripotent microvascular pericytes are the adult stem cells even in the testis. Adv. Exp. Med. Biol. 2019, 1122, 235-267.
12. Lv, F.-J.; Tuan, R. S.; Cheung, K. M. C.; Leung, V. Y. L. Concise review: The surface markers and identity of human mesenchymal stem cells: Markers and identity of MSCs. Stem Cells 2014, 32, 1408-1419.
13. Crisan, M.; Corselli, M.; Chen, W. C. W.; Péault, B. Perivascular cells for regenerative medicine. J. Cell. Mol. Med. 2012, 16, 2851-2860.
14. Sá da Bandeira, D.; Casamitjana, J.; Crisan, M. Pericytes, integral components of adult hematopoietic stem cell niches. Pharmacol. Ther. 2017, 171, 104-113.
15. Sims, D. E. The pericyte-A review. Tissue Cell 1986, 18, 153-174.
16. Mannino, G.; Gennuso, F.; Giurdanella, G.; Conti, F.; Drago, F.; Salomone, S.; Furno, D. L.; Bucolo, C.; Giuffrida, R. Pericyte-like differentiation of human adipose-derived mesenchymal stem cells: An in vitro study. World J. Stem Cells 2020, 12, 1152-1170.
17. Binamé, F. Transduction of extracellular cues into cell polarity: The role of the transmembrane proteoglycan NG2. Mol. Neurobiol. 2014, 50, 482-493.
18. Barilani, M.; Peli, V.; Cherubini, A.; Dossena, M.; Dolo, V.; Lazzari, L. NG2 as an identity and quality marker of mesenchymal stem cell extracellular vesicles. Cells 2019, 8, 1524.
19. Loder, S. J.; Agarwal, S.; Chung, M. T.; Cholok, D.; Hwang, C.; Visser, N.; Vasquez, K.; Sorkin, M.; Habbouche, J.; Sung, H. H.; et al. Characterizing the circulating cell populations in traumatic heterotopic ossification. Am. J. Pathol. 2018, 188, 2464-2473.
20. Naaijkens, B. A.; Krijnen, P. A.; Meinster, E.; ter Horst, E. N.; Vo, K.; Musters, R. J.; Kamp, O.; Niessen, H. W.; Juffermans, L. J.; van Dijk, A. Acute myocardial infarction does not affect functional characteristics of adipose-derived stem cells in rats, but reduces the number of stem cells in adipose tissue. Cell Tissue Res. 2005, 362, 623-632.
21. Díaz-Flores, L.; Gutiérrez, R.; Varela, H.; Rancel, N.; Valladares, F. Microvascular pericytes: A review of their morphological and functional characteristics. Histol. Histopathol. 1991, 6, 269-286.
22. Guillemin, G. J.; Brew, B. J. Microglia, macrophages, perivascular macrophages, and pericytes: A review of function and identification. J. Leukoc. Biol. 2004, 75, 388-397.
23. Fernandez-Klett, F.; Priller, J. Diverse functions of pericytes in cerebral blood flow regulation and ischemia. J. Cereb. Blood Flow Metab. 2015, 35, 883-88760.
24. Tillet, E.; Ruggiero, F.; Nishiyama, A.; Stallcup, W. B. The membrane-spanning proteoglycan NG2 binds to collagens V and VI through the central nonglobular domain of its core protein. J. Biol. Chem. 1997, 272, 10769-10776.
25. Kuo, H.-J.; Maslen, C. L.; Keene, D. R.; Glanville, R. W. Type VI collagen anchors endothelial basement membranes by interacting with Type IV collagen. J. Biol. Chem. 1997, 272, 26522-26529.
26. Ratajczak, M. Z.; Ratajczak, J.; Suszynska, M.; Miller, D. M.; Kucia, M.; Shin, D. M. A novel view of the adult stem cell compartment from the perspective of a quiescent population of very small embryonic-like stem cells. Circ. Res. 2017, 120, 166-178.
27. Ratajczak, M. Z.; Ratajczak, J.; Kucia, M. Very small embryonic-like stem cells (VSELs). Circ. Res. 2019, 124, 208-210.
28. Song, Y. H.; Prantl, L.; Alt, E. Differentiation and plasticity of stem cells for tissue engineering. In Tissue Engineering; Pallua, N., Suscheck, C. V., Eds.; Springer: Berlin, Germany, 2011; pp. 113-130.
29. Bruno, I.; Martinez, R.; Sanchez, A.; Friddle, C.; McClure, S. R.
Characterization of nucleated cells from equine adipose tissue and bone marrow aspirate processed for point-of-care use. J. Equine Vet. Sci. 2014, 34, 1118-1127.
30. Velten, L.; Haas, S. F.; Raffel, S.; Blaszkiewicz, S.; Islam, S.; Hennig, B. P.; Hirche, C.; Lutz, C.; Buss, E. C.; Nowak, D.; et al. Human haematopoietic stem cell lineage commitment is a continuous process. Nat. Cell Biol. 2017, 19, 271-281.
31. Schmitz, C.; Alt, E. Stammzellentherapie. Erstmalige Erfolgreiche Behandlung von Teilrupturen der Rotatorenmanschette Mit Frischen, Adulten, Körpereigenen Regenerativen Zellen. Available online: https://sportaerztezeitung.com/rubriken/therapie/1227/stammzellentherapie/(accessed on 30 Aug. 2021).
32. Di Russo, J.; Hannocks, M. J.; Luik, A. L.; Song, J.; Zhang, X.; Yousif, L.; Aspite, G.; Hallmann, R.; Sorokin, L. Vascular laminins in physiology and pathology. Matrix Biol. 2017, 57-58, 140-148.
33. Suzuki, S.; Namiki, J.; Shibata, S.; Mastuzaki, Y.; Okano, H. The neural stem/progenitor cell marker nestin is expressed in proliferative endothelial cells, but not in mature vasculature. J. Histochem. Cytochem. 2010, 58, 721-730.
34. Clark, R. A. F. Fibronectin matrix deposition and fibronectin receptor expression in healing and normal skin. J. Investig. Dermatol. 1990, 94, 128S-134S.
35. Serini, G.; Valdembri, D.; Bussolino, F. Integrins and angiogenesis: A sticky business. Exp. Cell Res. 2006, 312, 651-658. [CrossRef]
36. Vega, M. E.; Schwarzbauer, J. E. Collaboration of fibronectin matrix with other extracellular signals in morphogenesis and differentiation. Curr. Opin. Cell Biol. 2016, 42, 1-6.
37. Lane, T. F.; Sage, E. H. The biology of SPARC, a protein that modulates cell-matrix interactions. FASEB J. 1994, 8, 163-173.
38. Murphy-Ullrich, J. E.; Sage, E. H. Revisiting the matricellular concept. Matrix Biol. 2014, 37, 1-14.
39. Nie, J.; Chang, B.; Traktuev, D. O.; Sun, J.; March, K.; Chan, L.; Sage, E. H.; Pasqualini, R.; Arap, W.; Kolonin, M. G. IFATS collection: Combinatorial peptides identify alpha5beta1 integrin as a receptor for the matricellular protein SPARC on adipose stromal cells. Stem Cells 2008, 26, 2735-2745.
40. Tseng, C.; Kolonin, M. G. Proteolytic isoforms of SPARC induce adipose stromal cell mobilization in obesity: Mobilization of adipose stromal cells by SPARC. Stem Cells 2016, 34, 174-190.
41. Holland, P. W.; Harper, S. J.; McVey, J. H.; Hogan, B. L. In vivo expression of mRNA for the Ca++-binding protein SPARC (osteonectin) revealed by in situ hybridization. J. Cell Biol. 1987, 105, 473-482.
42. Brekken, R. A.; Sage, E. H. SPARC, a matricellular protein: At the crossroads of cell-matrix communication. Matrix Biol. 2001, 19, 815-827.
43. Chiodoni, C.; Colombo, M. P.; Sangaletti, S. Matricellular proteins: From homeostasis to inflammation, cancer, and metastasis. Cancer Metastasis Rev. 2010, 29, 295-307.
44. Bradshaw, A. D. The role of secreted protein acidic and rich in cysteine (SPARC) in cardiac repair and fibrosis: Does expression of SPARC by macrophages influence outcomes? J. Mol. Cell. Cardiol. 2016, 93, 156-161.
45. Le Bail, B.; Faouzi, S.; Boussarie, L.; Guirouilh, J.; Blanc, J. F.; Carles, J.; Bioulac-Sage, P.; Balabaud, C.; Rosenbaum, J. Osteonectin/SPARC is overexpressed in human hepatocellular carcinoma. J. Pathol. 1999, 189, 46-52.
46. Shaik, S.; Wu, X.; Gimble, J.; Devireddy, R. Effects of decade long freezing storage on adipose derived stem cells functionality. Sci. Rep. 2018, 8, 8162.
47. Choi, Y. A.; Lim, J.; Kim, K. M.; Acharya, B.; Cho, J. Y.; Bae, Y. C.; Shin, H. I.; Kim, S. Y.; Park, E. K. Secretome analysis of human BMSCs and identification of SMOC1 as an important ECM protein in osteoblast differentiation. J. Proteome Res. 2010, 9, 2946-2956.
48. Kusuma, G. D.; Carthew, J.; Lim, R.; Frith, J. E. Effect of the microenvironment on mesenchymal stem cell paracrine signaling: Opportunities to engineer the therapeutic effect. Stem Cells Dev. 2017, 26, 617-631.
49. Alt, E.; Yan, Y.; Gehmert, S.; Song, Y. H.; Altman, A.; Gehmert, S.; Vykoukal, D.; Bai, X. Fibroblasts share mesenchymal phenotypes with stem cells, but lack their differentiation and colony-forming potential. Biol. Cell 2011, 103, 197-208.
50. Holmes, C.; Stanford, W. L. Concise review: Stem cell antigen-1: Expression, function, and enigma. Stem Cells 2007, 25, 1339-1347.
51. D′Ippolito, G.; Howard, G. A.; Roos, B. A.; Schillerc, P. C. Sustained stromal stem cell self-renewal and osteoblastic differentiation during aging. Rejuvenation Res. 2006, 9, 10-19.
52. Corselli, M.; Chen, C.-W.; Sun, B.; Yap, S.; Rubin, J. P.; Péault, B. The tunica adventitia of human arteries and veins as a source of mesenchymal stem cells. Stem Cells Dev. 2012, 21, 1299-1308.
53. de Souza, L. E. B.; Malta, T. M.; Kashima Haddad, S.; Covas, D. T. Mesenchymal stem cells and pericytes: To what extent are they related? Stem Cells Dev. 2016, 25, 1843-1852.
54. Ilmer, M.; Vykoukal, J.; Boiles, A. R.; Coleman, M.; Alt, E. Two sides of the same coin: Stem cells in cancer and regenerative medicine. FASEB J. 2014, 28, 2748-2761.
55. Alt, E. U.; Winnier, G.; Haenel, A.; Rothoerl, R.; Solakoglu, O.; Alt, C.; Schmitz, C. Towards a comprehensive understanding of UA-ADRCs (uncultured, autologous, fresh, unmodified, adipose derived regenerative cells, isolated at point of care) in regenerative medicine. Cells 2020, 9, 1097.
56. Willert, K.; Brown, J. D.; Danenberg, E.; Duncan, A. W.; Weissman, I. L.; Reya,
T.; Yates, J. R.; Nusse, R. Wnt proteins are lipidmodified and can act as stem cell growth factors. Nature 2003, 423, 448-452.
57. Coudreuse, D.; Korswagen, H. C. The making of Wnt: New insights into Wnt maturation, sorting and secretion. Development 2007, 134, 3-12.
58. Neth, P.; Ries, C.; Karow, M.; Egea, V.; Ilmer, M.; Jochum, M. The Wnt signal transduction pathway in stem cells and cancer cells: Influence on cellular invasion. Stem Cell Rev. 2007, 3, 18-29.
59. Bourin, P.; Bunnell, B. A.; Casteilla, L.; Dominici, M.; Katz, A. J.; March, K. L.; Redl, H.; Rubin, J. P.; Yoshimura, K.; Gimble, J. M. Stromal cells from the adipose tissue-derived stromal vascular fraction and culture expanded adipose tissue-derived stromal/stem cells: A joint statement of the International Federation for Adipose Therapeutics and Science (IFATS) and the International Society for Cellular Therapy (ISCT). Cytotherapy 2013, 15, 641-648.
60. Rezza, A.; Sennett, R.; Rendl, M. Adult stem cell niches: Cellular and molecular components. Curr. Top. Dev. Biol. 2014, 107, 333-372.
61. Dong, L.; Hao, H.; Han, W.; Fu, X. The Role of the microenvironment on the fate of adult stem cells. Sci. China Life Sci. 2015, 58, 639-648.
62. Wabik, A.; Jones, P. H. Switching roles: The functional plasticity of adult tissue stem cells. EMBO J. 2015, 34, 1164-1179.
63. Ader, M.; Tanaka, E. M. Modeling human development in 3D culture. Curr. Opin. Cell Biol. 2014, 31, 23-28.
64. Izadpanah, R.; Trygg, C.; Patel, B.; Kriedt, C.; Dufour, J.; Gimble, J. M.; Bunnell, B. A. Biologic properties of mesenchymal stem cells derived from bone marrow and adipose tissue. J. Cell. Biochem. 2006, 99, 1285-1297.
65. Mitchell, J. B.; McIntosh, K.; Zvonic, S.; Garrett, S.; Floyd, Z. E.; Kloster, A.; Di Halvorsen, Y.; Storms, R. W.; Goh, B.; Kilroy, G.; et al. Immunophenotype of human adipose-derived cells: Temporal changes in stromal-associated and stem cell-associated markers. Stem Cells 2006, 24, 376-385.
66. Valente, S.; Alviano, F.; Ciavarella, C.; Buzzi, M.; Ricci, F.; Tazzari, P.; Pagliaro, P.; Pasquinelli, G. Human cadaver multipotent stromal/stem cells isolated from arteries stored in liquid nitrogen for 5 years. Stem Cell Res. Ther. 2014, 5, 8.
67. Bai, X.; Sadat, S.; Gehmert, S.; Alt, E.; Song, Y. H. VEGF Receptor Flk-1 plays an important role in c-Kit expression in adipose tissue derived stem cells. FEBS Lett. 2007, 581, 4681-4684. [CrossRef] [PubMed]
68. Metzele, R.; Alt, C.; Bai, X.; Yan, Y.; Zhang, Z.; Pan, Z.; Coleman, M.; Vykoukal, J.; Song, Y.; Alt, E. Human adipose tissue-derived stem cells exhibit proliferation potential and spontaneous rhythmic contraction after fusion with neonatal rat cardiomyocytes. FASEB J. 2011, 25, 830-839.
69. Belema Bedada, F.; Technau, A.; Ebelt, H.; Schulze, M.; Braun, T. Activation of myogenic differentiation pathways in adult bone marrow-derived stem cells. Mol. Cell. Biol. 2005, 25, 9509-9519.
70. Haenel, A.; Ghosn, M.; Karimi, T.; Vykoukal, J.; Shah, D.; Valderrabano, M.; Schulz, D. G.; Raizner, A.; Schmitz, C.; Alt, E. U. Unmodified autologous stem cells at point of care for chronic myocardial infarction. World J. Stem Cells 2019, 11, 831-858.
71. Yukawa, H.; Mizufune, S.; Mamori, C.; Kagami, Y.; Oishi, K.; Kaji, N.; Okamoto, Y.; Takeshi, M.; Noguchi, H.; Baba, Y.; et al. Quantum dots for labeling adipose tissue-derived stem cells. Cell Transpl. 2009, 18, 591-600.
72. Brinkley, B. R.; Beall, P. T.; Wible, L. J.; Mace, M. L.; Turner, D. S.; Cailleau, R. M. Variations in cell form and cytoskeleton in human breast carcinoma cells in vitro. Cancer Res. 1980, 40, 3118-3129.
73. Koyanagi, M.; Brandes, R. P.; Haendeler, J.; Zeiher, A. M.; Dimmeler, S. Cell-to-cell connection of endothelial progenitor cells with cardiac myocytes by nanotubes: A novel mechanism for cell fate changes? Circ. Res. 2005, 96, 1039-1041.
74. Valadi, H.; Ekström, K.; Bossios, A.; Sjöstrand, M.; Lee, J. J.; Lötvall, J. O. Exosome-mediated transfer of mRNAs and microRNAs is a novel mechanism of genetic exchange between cells. Nat. Cell Biol. 2007, 9, 654-659.
75. Xin, H.; Li, Y.; Buller, B.; Katakowski, M.; Zhang, Y.; Wang, X.; Shang, X.; Zhang, Z. G.; Chopp, M. Exosome-mediated transfer of miR-133b from multipotent mesenchymal stromal cells to neural cells contributes to neurite outgrowth. Stem Cells 2012, 30, 1556-1564.
76. Ismail, N.; Wang, Y.; Dakhlallah, D.; Moldovan, L.; Agarwal, K.; Batte, K.; Shah, P.; Wisler, J.; Eubank, T. D.; Tridandapani, S.; et al. Macrophage microvesicles induce macrophage differentiation and miR-223 transfer. Blood 2013, 121, 984-995.
77. Kruger, S.; Elmageed, Z. Y. A.; Hawke, D. H.; Wörner, P. M.; Jansen, D. A.; Abdel-Mageed, A. B.; Alt, E. U.; Izadpanah, R. Molecular characterization of exosome-like vesicles from breast cancer cells. BMC Cancer 2014, 14, 44.
78. Xu, J. Y.; Chen, G. H.; Yang, Y. J. Exosomes: A rising star in failing hearts. Front. Physiol. 2017, 8, 494.
79. González, M. A.; Gonzalez-Rey, E.; Rico, L.; Büscher, D.; Delgado, M. Adipose-derived mesenchymal stem cells alleviate experimental colitis by inhibiting inflammatory and autoimmune responses. Gastroenterology 2009, 136, 978-989.
80. Leto Barone, A. A.; Khalifian, S.; Lee,W. P. A.; Brandacher, G. Immunomodulatory effects of adipose-derived stem cells: Fact or fiction? BioMed Res. Int. 2013, 2013, 383685.
81. Caruso, S.; Poon, I. K. H. Apoptotic cell-derived extracellular vesicles: More than just debris. Front. Immunol. 2018, 9, 1486.
82. Galleu, A.; Riffo-Vasquez, Y.; Trento, C.; Lomas, C.; Dolcetti, L.; Cheung, T. S.; von Bonin, M.; Barbieri, L.; Halai, K.; Ward, S.; et al. Apoptosis in mesenchymal stromal cells induces in vivo recipient-mediated immunomodulation. Sci. Transl. Med. 2017, 9, eaam7828.
83. Hochreiter-Hufford, A.; Ravichandran, K. S. Clearing the dead: Apoptotic cell sensing, recognition, engulfment, and digestion. Cold Spring Harb. Perspect. Biol. 2013, 5, a008748.
84. Lauber, K.; Bohn, E.; Kröber, S. M.; Xiao, Y.; Blumenthal, S. G.; Lindemann, R. K.; Marini, P.; Wiedig, C.; Zobywalski, A.; Baksh, S.; et al. Apoptotic cells induce migration of phagocytes via caspase-3-mediated release of a lipid attraction signal. Cell 2003, 113, 717-730.
85. Fuchs, B.; Schiller, J.; Cross, M. A. Apoptosis-associated changes in the glycerophospholipid composition of hematopoietic progenitor cells monitored by 31P NMR spectroscopy and MALDI-TOF mass spectrometry. Chem. Phys. Lipids 2007, 150, 229-238.
86. Rakic, P. DNA Synthesis and cell division in the adult primate brain. Ann. N. Y. Acad. Sci. 1985, 457, 193-211.
87. Breunig, J. J.; Arellano, J. I.; Macklis, J. D.; Rakic, P. Everything that glitters isn't gold: A critical review of postnatal neural precursor analyses. Cell Stem Cell 2007, 1, 612-627.
88. Jellinger, K. A.; Attems, J. Neuropathological approaches to cerebral aging and neuroplasticity. Dialogues Clin. Neurosci. 2013, 15, 29-43.
89. Demidova-Rice, T. N.; Hamblin, M. R.; Herman, I. M. Acute and impaired wound healing: Pathophysiology and current methods for drug delivery, part 1: Normal and chronic wounds: Biology, causes, and approaches to care. Adv. Skin Wound Care 2012, 25, 304-314.
90. McCusker, C.; Gardiner, D. M. The axolotl model for regeneration and aging research: A mini-review. Gerontology 2011, 57, 565-571.
91. Farah, Z.; Fan, H.; Liu, Z.; He, J. Q. A concise review of common animal models for the study of limb regeneration. Organogenesis 2016, 12, 109-118.
92. Grillo, M.; Konstantinides, N.; Averof, M. Old questions, new models: Unraveling complex organ regeneration with new experimental approaches. Curr. Opin. Genet. Dev. 2016, 40, 23-31.
93. Zielins, E. R.; Ransom, R. C.; Leavitt, T. E.; Longaker, M. T.; Wan, D. C. The role of stem cells in limb regeneration. Organogenesis 2016, 12, 16-27.
94. Satoh, A.; Mitogawa, K.; Makanae, A. Regeneration inducers in limb regeneration. Dev. Growth Differ. 2015, 57, 421-429.
95. Haas, B. J.; Whited, J. L. Advances in decoding axolotl limb regeneration. Trends Genet. 2017, 33, 553-565.
96. Jullien, J.; Pasque, V.; Halley-Stott, R. P.; Miyamoto, K.; Gurdon, J. B. Mechanisms of nuclear reprogramming by eggs and oocytes: A deterministic process? Nat. Rev. Mol. Cell Biol. 2011, 12, 453-459.
97. Heffernan, C.; Sumer, H.; Verma, P. J. Generation of clinically relevant ‘induced pluripotent stem” (iPS) cells. J. Stem Cells 2011, 6, 109-127.
98. Shi, Y.; Inoue, H.; Wu, J. C.; Yamanaka, S. Induced pluripotent stem cell technology: A decade of progress. Nat. Rev. Drug Discov. 2017, 16, 115-130.
99. Takahashi, K.; Tanabe, K.; Ohnuki, M.; Narita, M.; Ichisaka, T.; Tomoda, K.; Yamanaka, S. Induction of pluripotent stem cells from adult human fibroblasts by defined factors. Cell 2007, 131, 861-872.
100. Yu, J.; Vodyanik, M. A.; Smuga-Otto, K.; Antosiewicz-Bourget, J.; Frane, J. L.; Tian, S.; Nie, J.; Jonsdottir, G. A.; Ruotti, V.; Stewart, R.; et al. Induced pluripotent stem cell lines derived from human somatic cells. Science 2007, 318, 1917-1920.
101. Lee, A. S.; Tang, C.; Rao, M. S.; Weissman, I. L.; Wu, J. C. Tumorigenicity as a clinical hurdle for pluripotent stem cell therapies. Nat. Med. 2013, 19, 998-1004.
102. Riggs, J. W.; Barrilleaux, B. L.; Varlakhanova, N.; Bush, K. M.; Chan, V.; Knoepfler, P. S. Induced pluripotency and oncogenic transformation are related processes. Stem Cells Dev. 2013, 22, 37-50.
103. Steinemann, D.; Göhring, G.; Schlegelberger, B. Genetic instability of modified stem cells-a first step towards malignant transformation? Am. J. Stem Cells 2013, 2, 39-51.
104. Tung, P.-Y.; Knoepfler, P. S. Epigenetic mechanisms of tumorigenicity manifesting in stem cells. Oncogene 2015, 34, 2288-2296.
105. Lee, A. S.; Tang, C.; Hong, W. X.; Park, S.; Bazalova-Carter, M.; Nelson, G.; Sanchez-Freire, V.; Bakerman, I.; Zhang, W.; Neofytou, E.; et al. Brief report: External beam radiation therapy for the treatment of human pluripotent stem cell-derived teratomas. Stem Cells 2017, 35, 1994-2000.
106. Marsoner, F.; Koch, P.; Ladewig, J. Cortical organoids: Why all this hype? Curr. Opin. Genet. Dev. 2018, 52, 22-28.
107. Murphy, M. B.; Moncivais, K.; Caplan, A. I. Mesenchymal stem cells: Environmentally responsive therapeutics for regenerative medicine. Exp. Mol. Med. 2013, 45, e54.
108. Al-Drees, M. A.; Yeo, J. H.; Boumelhem, B. B.; Antas, V. I.; Brigden, K. W. L.; Colonne, C. K.; Fraser, S. T. Making blood: The haematopoietic niche throughout ontogeny. Stem Cells Int. 2015, 2015, 571893.
109. Yoshimura, K.; Shigeura, T.; Matsumoto, D.; Sato, T.; Takaki, Y.; Aiba-Kojima, E.; Sato, K.; Inoue, K.; Nagase, T.; Koshima, I.; et al. Characterization of freshly isolated and cultured cells derived from the fatty and fluid portions of liposuction aspirates. J. Cell. Physiol. 2006, 208, 64-76.
110. Schäffler, A.; Büchler, C. Concise review: Adipose tissue-derived stromal cells-basic and clinical implications for novel cell-based therapies. Stem Cells 2007, 25, 818-827.
111. Gimble, J. M.; Guilak, F.; Bunnell, B. A. Clinical and preclinical translation of cell-based therapies using adipose tissue-derived cells. Stem Cell Res. Ther. 2010, 1, 19.
112. Altman, A. M.; Khalek, F. J. A.; Alt, E. U.; Butler, C. E. Adipose tissue-derived stem cells enhance bioprosthetic mesh repair of ventral hernias. Plast. Reconstr. Surg. 2010, 126, 845-854.
113. Gir, P.; Oni, G.; Brown, S. A.; Mojallal, A.; Rohrich, R. J. Human adipose stem cells: Current clinical applications. Plast. Reconstr. Surg. 2012, 129, 1277-1290.
114. Klein, S. M.; Vykoukal, J.; Li, D. P.; Pan, H. L.; Zeitler, K.; Alt, E.; Geis, S.; Felthaus, O.; Prantl, L. Peripheral motor and sensory nerve conduction following transplantation of undifferentiated autologous adipose tissue-derived stem cells in a biodegradable U. S. Food and Drug Administration approved nerve conduit. Plast. Reconstr. Surg. 2016, 138, 132-139.
115. Oberbauer, E.; Steffenhagen, C.; Wurzer, C.; Gabriel, C.; Redl, H.; Wolbank, S. Enzymatic and non-enzymatic isolation systems for adipose tissue-derived cells: Current state of the art. Cell Regen. 2015, 4, 7.
116. Conde-Green, A.; Kotamarti, V. S.; Sherman, L. S.; Keith, J. D.; Lee, E. S.; Granick, M. S.; Rameshwar, P. Shift toward mechanical isolation of adipose-derived stromal vascular fraction: Review of upcoming techniques. Plast. Reconstr. Surg. Glob. Open 2016, 4, e1017.
117. van Dongen, J. A.; Tuin, A. J.; Spiekman, M.; Jansma, J.; van der Lei, B.; Harmsen, M. C. Comparison of intraoperative procedures for isolation of clinical grade stromal vascular fraction for regenerative purposes: A systematic review: Intraoperative procedures for stromal vascular fraction isolation. J. Tissue Eng. Regen. Med. 2018, 12, e261-e274.
118. Metcalf, G. L.; McClure, S. R.; Hostetter, J. M.; Martinez, R. F.; Wang, C. Evaluation of adipose-derived stromal vascular fraction from the lateral tailhead, inguinal region, and mesentery of horses. Can. J. Vet. Res. 2016, 80, 294-301.
119. Aronowitz, J. A.; Ellenhorn, J. D. I. Adipose stromal vascular fraction isolation: A head-to-head comparison of four commercial cell separation systems. Plast. Reconstr. Surg. 2013, 132, 932e-939e.
120. Aronowitz, J. A.; Lockhart, R. A.; Hakakian, C. S.; Birnbaum, Z. E. Adipose stromal vascular fraction isolation: A head-to-head comparison of 4 cell separation systems #2. Ann. Plast. Surg. 2016, 77, 354-362.
121. de Almeida, P. E.; van Rappard, J. R. M.; Wu, J. C. In vivo bioluminescence for tracking cell fate and function. Am. J. Physiol. Heart Circ. Physiol. 2011, 301, H663-H671.
122. Scarfe, L.; Brillant, N.; Kumar, J. D.; Ali, N.; Alrumayh, A.; Amali, M.; Barbellion, S.; Jones, V.; Niemeijer, M.; Potdevin, S.; et al. Preclinical imaging methods for assessing the safety and efficacy of regenerative medicine therapies. NPJ Regen. Med. 2017, 2, 28.
123. Bai, X.; Yan, Y.; Coleman, M.; Wu, G.; Rabinovich, B.; Seidensticker, M.; Alt, E. Tracking long-term survival of intramyocaradially delivered human adipose tissue-derived stem cells using bioluminescence imaging. Mol. Imaging Biol. 2011, 13, 633-645.
124. Sadat, S.; Gehmert, S.; Song, Y.-H.; Yen, Y.; Bai, X.; Gaiser, S.; Klein, H.; Alt, E. The cardioprotective effect of mesenchymal stem cells is mediated by IGF-I and VEGF. Biochem. Biophys. Res. Commun. 2007, 363, 674-679.
125. Rothoerl, R.; Alt, C.; Preuss, A.; Mueller, C.; Lackermeier, P.; Alt, E. Clinical results of adipose derived stem cell injection for facet joint syndrome. In Proceedings of the IFATS San Diego 2016 Conference Proceedings, San Diego, CA, USA, 17-20 Nov. 2016; p. 79. Available online: https://www.ifats.org/assets/docs/IFATS-Program-Book-2016-FINAL-11.23.16.pdf (accessed on 6 Aug. 2021).
126. Konstantinow, A.; Arnold, A.; Djabali, K.; Kempf, W.; Gutermuth, J.; Fischer, T.; Biedermann, T. Therapy of ulcus cruris of venous and mixed venous arterial origin with autologous, adult, native progenitor cells from subcutaneous adipose tissue: A prospective clinical pilot study. J. Eur. Acad. Dermatol. Venereol. 2017, 31, 2104-2118.
127. Hurd, J. L.; Facile, T. R.; Weiss, J.; Hayes, M.; Hayes, M.; Furia, J. P.; Maffulli, N.; Winnier, G. E.; Alt, C.; Schmitz, C.; et al. Safety and efficacy of treating symptomatic, partial-thickness rotator cuff tear with fresh, uncultured, unmodified, autologous adipose derived regenerative cells (UA-ADRCs) isolated at the point of care: A prospective, randomized, controlled first-in-human pilot study. J. Orthop. Surg. Res. 2020, 15, 122.
128. Flier, J. S.; Underhill, L. H.; Dvorak, H. F. Tumors: Wounds that do not heal. N. Engl. J. Med. 1986, 315, 1650-1659. 129. Dvorak, H. F. Tumors: Wounds that do not heal-Redux. Cancer Immunol. Res. 2015, 3, 1-11.
130. Gehmert, S.; Gehmert, S.; Prantl, L.; Vykoukal, J.; Alt, E.; Song, Y.-H. Breast cancer cells attract the migration of adipose tissue-derived stem cells via the PDGF-BB/PDGFR-signaling pathway. Biochem. Biophys. Res. Commun. 2010, 398, 601-605.
131. Altman, A. M.; Yan, Y.; Matthias, N.; Bai, X.; Rios, C.; Mathur, A. B.; Song, Y. H.; Alt, E. U. IFATS collection: Human adipose-derived stem cells seeded on a silk fibroin-chitosan scaffold enhance wound repair in a murine soft tissue injury model. Stem Cells 2009, 27, 250-258.
132. Angst, F.; Schwyzer, H. K.; Aeschlimann, A.; Simmen, B. R.; Goldhahn, J. Measures of adult shoulder function: Disabilities of the Arm, Shoulder, and Hand Questionnaire (DASH) and its short version (QuickDASH), Shoulder Pain and Disability Index (SPADI), American Shoulder and Elbow Surgeons (ASES) Society standardized shoulder assessment form, Constant (Murley) Score (CS), Simple Shoulder Test (SST), Oxford Shoulder Score (OSS), Shoulder Disability Questionnaire (SDQ), andWestern Ontario Shoulder Instability Index (WOSI). Arthr. Care Res. 2011, 63 (Suppl. 11), S174-S188.
133. Hurd, J. Autologous Adult Adipose-Derived Regenerative Cell Injection into Chronic Partial-Thickness Rotator Cuff Tears. Available online: https://www.clinicaltrials.gov/ct2/show/NCT03752827 (accessed on 6 Aug. 2021).
134. Alt, E. U.; Rothoerl, R.; Hoppert, M.; Frank, H. G.; Wuerfel, T.; Alt, C.; Schmitz, C. First immunohistochemical evidence of human tendon repair following stem cell injection: A case report and review of literature. World J. Stem Cells 2021, 13, 944-970.
135. Shukunami, C.; Oshima, Y.; Hiraki, Y. Molecular Cloning of Tenomodulin, a Novel Chondromodulin-I Related Gene. Biochem. Biophys. Res. Commun. 2001, 280, 1323-1327.
136. Brandau, O.; Meindl, A.; Fässler, R.; Aszódi, A. A novel gene, Tendin, is strongly expressed in tendons and ligaments and shows high homology with Chondromodulin-I: Tendin and Chondromodulin-I. Dev. Dyn. 2001, 221, 72-80.
137. Dex, S.; Lin, D.; Shukunami, C.; Docheva, D. TENOgenic MODULating INsider factor: Systematic assessment on the functions of tenomodulin gene. Gene 2016, 587, 1-17.
138. Jung, C. K. Articular cartilage: Histology and physiology. In Techniques in Cartilage Repair Surgery; Shetty, A. A., Kim, S. J., Nakamura, N., Brittberg, M., Eds.; Springer: Berlin, Germany, 2014; pp. 17-21.
139. Rieppo, J.; Hallikainen, J.; Jurvelin, J. S.; Kiviranta, I.; Helminen, H. J.; Hyttinen, M. M. Practical considerations in the use of polarized light microscopy in the analysis of the collagen network in articular cartilage. Microsc. Res. Tech. 2008, 71, 279-287.
140. Damia, E.; Chicharro, D.; Lopez, S.; Cuervo, B.; Rubio, M.; Sopena, J.; Vilar, J.; Carrillo, J. Adipose-derived mesenchymal stem cells: Are they a good therapeutic strategy for osteoarthritis? Int. J. Mol. Sci. 2018, 19, 1926.
141. Jo, C. H.; Lee, Y. G.; Shin, W. H.; Kim, H.; Chai, J. W.; Jeong, E. C.; Kim, J. E.; Shim, H.; Shin, J. S.; Shin, I. S.; et al. Intra-articular injection of mesenchymal stem cells for the treatment of osteoarthritis of the knee: A proof-of-concept clinical trial: Ia injection of MSCs for knee osteoarthritis. Stem Cells 2014, 32, 1254-1266.
142. Pers, Y.-M.; Rackwitz, L.; Ferreira, R.; Pullig, O.; Delfour, C.; Barry, F.; Sensebe, L.; Casteilla, L.; Fleury, S.; Bourin, P.; et al. Adipose mesenchymal stromal cell-based therapy for severe osteoarthritis of the knee: A phase I dose-escalation trial: ASCs for severe OA of the knee. Stem Cells Transl. Med. 2016, 5, 847-856.
143. Freitag, J.; Li, D.; Wickham, J.; Shah, K.; Tenen, A. Effect of autologous adipose-derived mesenchymal stem cell therapy in the treatment of a post-traumatic chondral defect of the knee. BMJ Case Rep. 2017, 2017, bcr2017220852.
144. Alexander, C. E.; Sandean, D. P.; Varacallo, M. Lumbosacral facet syndrome. In StatPearls; StatPearls Publishing: Treasure Island, FL, USA, 2021.
145. Boetel, T. Safety of Adipose-Derived Regenerative Cells Injection for Treatment of Osteoarthritis of the Facet Joint. Available online: https://www.clinicaltrials.gov/ct2/show/NCT03513731 (accessed on 26 Jul. 2021).
146. Solakoglu, Ö.; Götz, W.; Kiessling, M. C.; Alt, C.; Schmitz, C.; Alt, E. U. Improved guided bone regeneration by combined application of unmodified, fresh autologous adipose derived regenerative cells and plasma rich in growth factors: A first-inhuman case report and literature review. World J. Stem Cells 2019, 11, 124-146.
147. Larson, E.; Jones, L. C.; Goodman, S. B.; Koo, K.-H.; Cui, Q. Early-stage osteonecrosis of the femoral head: Where are we and where are we going in year 2018? Int. Orthop. 2018, 42, 1723-1728.
148. Andriolo, L.; Merli, G.; Tobar, C.; Altamura, S. A.; Kon, E.; Filardo, G. Regenerative therapies increase survivorship of avascular necrosis of the femoral head: A systematic review and meta-analysis. Int. Orthop. 2018, 42, 1689-1704.
149. Pak, J.; Lee, J. H.; Jeon, J. H.; Lee, S. H. Complete resolution of avascular necrosis of the human femoral head treated with adipose tissue-derived stem cells and platelet-rich plasma. J. Int. Med. Res. 2014, 42, 1353-1362.
150. Sorice, S.; Rustad, K. C.; Li, A. Y.; Gurtner, G. C. The role of stem cell therapeutics in wound healing: Current understanding and future directions. Plast. Reconstr. Surg. 2016, 138, 31S-41S.
151. Altman, A. M.; Abdul Khalek, F. J.; Seidensticker, M.; Pinilla, S.; Yan, Y.; Coleman, M.; Song, Y.-H.; Butler, C. E.; Alt, E. U. Human tissue-resident stem cells combined with hyaluronic acid gel provide fibrovascular-integrated soft-tissue augmentation in a murine photoaged skin model. Plast. Reconstr. Surg. 2010, 125, 63-73.
152. Pourjafar, M.; Saidijam, M.; Etemadi, K.; Najafi, R. All-trans retinoic acid enhances in vitro mesenchymal stem cells migration by targeting matrix metalloproteinases 2 and 9. Biotechnol. Lett. 2017, 39, 1263-1268.
153. Master, Z.; Matthews, K. R. W.; Abou-el-Enein, M. Unproven stem cell interventions: A global public health problem requiring global deliberation. Stem Cell Rep. 2021, 16, 1435-1445.

1. A method of treating a defect in a mammalian subject, comprising:
a) isolating unmodified autologous vascular-associated pluripotent stem cells (UA-vaPS) from a mammalian subject;
b) introducing the UA-vaPS into the mammalian subject at or near the defect.

2. The method of claim 1, wherein the UA-vaPS are isolated from a vascular tissue from the mammalian subject.

3. The method of claim 1, wherein the UA-vaPS are isolated from an adipose-tissue from the mammalian subject.

4. The method of claim 1, wherein the UA-vaPS are not cultured or expanded in vitro prior to step b).

5. The method of claim 1, wherein the defect includes at least one of: tendon defects, cartilage defects, chronic, recalcitrant low back pain caused by lumbosacral facet syndrome, avascular necrosis of femoral head, wounds, scar tissues, and hair loss.
